(19) United States
(12) Patent Application Publication
(10) Pub. No.: US 2010/0311157 A1
(43) Pub. Date: Dec. 9, 2010

(54) SYSTEMS AND METHODS FOR PRODUCTION OF BIOFUEL

(76) Inventors: David Van Alstyne, St. Cyrus (GB); Laylah Elizabeth Van Alstyne, Cyrus (GB)

Correspondence Address: BELL & ASSOCIATES, 58 West Portal Avenue No. 121, SAN FRANCISCO, CA, 94127, US

(21) Appl. No.: 12/742,254

(22) PCT Filed: Nov. 13, 2008
(86) PCT No.: PCT/IB08/03048

§ 371 Date: Aug. 23, 2010

Related U.S. Application Data
(60) Provisional application No. 61/132,290, filed on Jun. 16, 2008.
(60) Provisional application No. 61/002,856, filed on Nov. 13, 2007.

(51) Int. Cl. C12M 1/04

(52) U.S. Cl. 435/294.1

(57) ABSTRACT

Self-sustaining, self-contained systems and methods for producing biofuels and for producing biofuel feedstock from algae. The system is carbon neutral or may be carbon positive, fixing more carbon than it releases to the atmosphere. In various embodiments, the system may be coupled to an existing carbon dioxide producing process to reduce or completely eliminate carbon dioxide output, making the existing system carbon neutral, and providing valuable and tradable carbon credits. The system may also comprise modular tiles comprising a biomass sandwiched between two panels and use a combination of microbes, nutrients, water, and sunlight to generate biological hydrocarbon compounds that can be used in almost any type of engine.

The present application claims priority to U.S. Provisional Patent Application Ser. No. 61/002,856 entitled “Systems And Methods For Production Of Biofuel”, filed Nov. 13, 2007, and U.S. Provisional Patent Application Ser. No. 61/132,290 entitled “Systems And Methods For Production Of Biofuel”, filed Jun. 16, 2008, which are herein incorporated by reference in their entirety for all purposes.

### FIELD OF THE INVENTION

The inventive subject matter relates to self-contained systems and methods for producing biofuels and for producing biofuel feedstock from algae. In addition, the disclosure provides for a system of modular tiles comprising such self-contained systems and methods that may be placed upon or form part of the structure of a building.

### BACKGROUND

The majority of the energy requirements for the world economy is provided by burning fossil fuels. The fossil fuels are the primarily the remains of biological organisms that incorporated energy from the Sun using photosynthesis as well as the organisms that fed upon them. Under anaerobic conditions, such as a terrestrial burial under water, such as in a swamp or as pelagic remains in the oceans or shallow seas, the organisms' remains were not significantly degraded by bacteria and fungi to smaller molecules and thereby recycled into the biosphere. Over time, as the remains were overlain by successive layers of the remains of more organisms, or as the marine environment dried up to leave a crust of salts above, or as tectonic effects caused new sediments to be overlain, and under high pressure below the surface of the earth, the carbon-based compounds of the organism, such as sugars, amino acids, lipids, etc., underwent chemical and physical changes that eliminated oxygen and nitrogen, leaving a variety of hydrocarbons of varying length.

These hydrocarbon fuels are presently mined as coal, oil, and natural gas, a process that can incur not only energy costs, but significant use of other natural resources, such as minerals and ores, and, frequently, can lead to environmental damage. Competition for access to energy resources between the developed nations and the developing nations is expected to become intense during the early part of the 21^{st} Century and so alternative sources of energy must be exploited. To date, renewable energy, such as solar power, wind energy, and wave energy, have been used with some success to deliver energy, as electrical power, to users, such as industry and urbanizations. However, these sources are dependent upon the environmental conditions, and so electricity generation is unpredictable and intermittent. In addition, efficiencies making the se processes economically viable are generally only achieved through large capacity power generation; hence the facilities that house these renewable generators must be large and require significant regular maintenance.

For travel and transport, most vehicles use petroleum products, although there is an increasing demand (in the developed nations) for vehicles powered by electricity, hybrids, or fuel cells in order to reduce pollution into the local environment from burning the fossil fuels into the dependency upon hydrocarbon fossil fuels. Nevertheless, the electricity is usually generated by combustion of fossil fuels at a distant location, thus still contributing to environmental pollution as a whole.

Another fuel for vehicles (internal combustion engine) currently in use and slated for a significant increased in production and marketing, is ethanol. Many states in the U.S. are beginning to view mandating increased use of ethanol in place of fossil, fuels. A prime disadvantage cited against the production of ethanol is that it requires almost the same amount of energy input to produce it (including the transportation from still to distribution outlet) as it saves from producing fossil fuels.

Biodiesel (a fatty acid methyl ester) is a fuel produced from renewable resources like vegetable oil rather than petroleum and can be directly used as a fuel or blended with conventional diesel fuel made from petroleum (petrodiesel). Biodiesel can run in almost any vehicle that can run on petrodiesel with few or no modifications.

Most biodiesel is generally made in a batch process by mixing vegetable oil with methanol and exposing the mixture to a catalyst at elevated temperatures, letting the mixture settle, separating the products into biodiesel, glycerin and “soap,” washing the biodiesel with an acid/water solution, and finally removing the water from the cleaned biodiesel.

Biofuels, such as biodiesel, can be extracted relatively cheaply from cooking oils and fats, such as canola oil, but the original starting compositions have already used a significant amount of other energy resources to produce, for example, fertilizers, pesticides, tractors and trailers, harvesting, vegetable pulping, oil extraction, packaging, marketing, and transportation from field to kitchen.

The process of making biodiesel is a base catalyzed transesterification of a triglyceride. The ingredients used are generally are vegetable oil and methanol (or ethanol, etc.) and sodium hydroxide as the base:

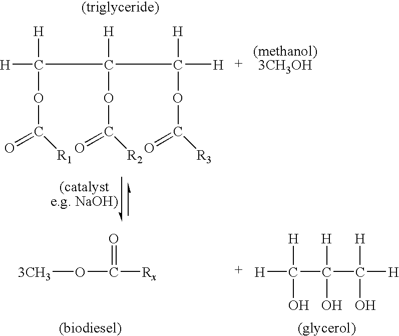

A summary of the steps is:
1) Coarse filtration of oil and drainage of any water present
(2) Sample oil and perform titration—determine quantity of catalyst
(3) Measure the reactants
(4) Dissolve NaOH into methanol
(5) Mix the reactants
(6) Allow glycerol to settle
(7) Drain glycerol
(8) Further processing for example, washing/drying/additives
(9) Filtration of biodiesel

Many substrates currently thought of as waste products may potentially be processed using microbiological processes to produce various types of biofuel. For example, excrement from farm animals can be used. This is in plentiful supply particularly when the animals are housed in concentrations of large numbers, for example, cattle lots or chicken farms. It has been realized that such animal (and human) waste has potential as an energy resource, but present methods have generally been considered too costly or uncompetitive for commercial application.

EN 14214 is an international standard that describes the minimum requirements for biodiesel that has been produced from virgin rapeseed fuel stock (also known as R.M.E. or rapeseed methyl esters).

The production of biodiesel from algae has been investigated by various groups including Michael Briggs at the University of New Hampshire and at the National Renewable Energy Laboratory (NREL) in Golden, Colo.

Bioreactors have been developed that can be used to treat such wastes. For example, U.S. Pat. No. 5,227,136 discloses a bioreactor vessel comprising a tank adapted to receive and contain a slurry, a mechanical mixing means fitted in the tank, an air supply means which involves the introduction of minute air bubbles near the bottom region of the tank by a plurality of elastic membrane diffusers (col. 3, line 20 to 32) and a means of re-circulating exhaust gas stream back into the reactor contained slurry by means of the diffusers (col. 4, line 6 to 11). In use, slurry containing minerals, soils and/or sludges which have been contaminated by toxic organic substances are delivered to the tank where they are directly contacted with and degraded by a biomass. Maintaining a high biomass concentration in the reactor is said to be a task requiring the use of equipment ancillary to the bioreactor (col. 4, lines 1 to 5) and in a preferred embodiment of the invention a biomass-carrying medium is added to the slurry contained in the tank to assist in maintaining a maximum biomass concentration (col. 10 lines 10 to 16).

U.S. Pat. No. 6,733,662 discloses using a bioreactor for the treatment of wastewater including residential, municipal and industrial wastewater. The devices and methods of the disclosed invention are useful for enhanced secondary wastewater treatment.

U.S. Pat. No. 6,244,038 describes a power plant with a fuel gas generator utilizing fluidized bed combustion.

U.S. Pat. No. 6,015,440 describes biodiesel production wherein triglycerides are reacted in a liquid phase reaction with methanol and a homogeneous basic catalyst to produce an upper phase of non-polar methyl esters and a lower phase or glycerol and residual methyl esters. The glycerol ethers are then added back to the upper located methyl ethyl ester phase to provide an improved biodiesel fuel.

In addition to the aforementioned publications, there are modular systems and methods that carry out all of the processes of solid state fermentation for the cultivation of micro-organisms, such as disclosed by Suryanarayan et al. in U.S. Pat. No. 6,664,095 in which heat generated by the bioreactor is specifically and deliberately removed from the system, in order to maintain a constant temperature for fermentation.

There remains a need for an energy resource that may be used as a fuel for vehicles, small engines, or electrical generators, that can be produced using few external resources, required no or little maintenance, and which can be operated and generated on a small scale. In addition, there remains a need for efficient disposal of organic waste, produced from human or farm animal excrement and/or kitchen waste whereby the system is compact enough for use by a single individual and that does not have the limitations of the current art.

### BRIEF SUMMARY OF THE INVENTION

The invention provides systems and methods for the production of biofuel from biomass. The biomass may be algal, or may be derived from any other source such as lignified or non-lignified plants or the extracts of plants or seeds, such as oils.

In one particular embodiment, the invention relates to self-sustaining, self-contained systems and methods for producing biofuels and for producing biofuel feedstock from algae. The system is carbon neutral or may be carbon positive, fixing more carbon than it releases to the atmosphere. Carbon dioxide is recycled through the algae to reduce carbon footprint. Additionally the system is self-powering and independent of external power output. Because the system is self-sustaining, it can be containerized, transported, and easily set up where needed.

In one particular embodiment the invention provides for an energy self-sufficient closed loop system that recycles waste byproducts of the biodiesel production process to provide energy to power the process. Other byproducts are used to produce economically useful products such as animal feed, fertilizer and fuel. An important feature of the invention is that it is carbon neutral or substantially carbon-neutral, or in some cases, actually carbon negative, that is, consuming and fixing more carbon (into biomass) than it releases into the atmosphere.

One exemplary embodiment, herein termed “The Symbiotic Digestor and Photobioreactor” system, uses organisms that use solar energy (for example algae or cyanobacteria) to produce covalent bonds between simple organic compounds (carbon dioxide). The Symbiotic Digestor and Photobioreactor is a 2-part closed system with one half of the system using yeast and a carbohydrate source to generate carbon dioxide gas and the other half using the carbon dioxide produced to provide a carbon source for the growing algal biomass. Any byproducts generated are re-used in the system.

In both closed loop and with the Symbiotic Digestor and Photobioreactor system, sea water may be used in which to grow the algae (and bacteria). This provides an additional benefit in that the systems require no fresh water. Either system may be containerized and located on land or at sea.

The systems disclosed herein can be used together so that the Symbiotic Digestor and Photobioreactor produces algal biomass that acts as feedstock for the closed loop system.

The lack of requirement of either system for an external energy source or for fresh water makes the system versatile, inexpensive and portable. Additionally very little maintenance is required. This makes the system ideal for poor economies or for situations in which resources, energy or land is in short supply.

In another preferred embodiment the system uses a tile comprising two plates (for example manufactured from PERSPEX/PLEXIGLASS or glass) held proximal to one another using a frame, the two plates and the frame defining a lumen therebetween, wherein the lumen can be filled to a desired capacity with a biological organism. In an alternative, the organism can be a modified biological organism further comprising at least one synthetic gene that provides for synthesis of a protein or other organic compound that results in an increased level of total measured energy for a given mass of the organism, when compared with the energy of a similar, non-modified organism. In another embodiment, the modified biological organism comprises at least one synthetic biological pathway that results in an increased level of total measured energy for a given mass of the organism, when compared with the energy of a similar, non-modified organism.

### BRIEF DESCRIPTION OF THE FIGURES

FIG. 1 illustrates an exemplary closed loop bioreactor.

FIG. 2 illustrates an exemplary paired bioreactor (“Symbiotic Digestor and Photobioreactor”) system acting in symbiosis.

FIG. 3 illustrates a tile comprising two PERSPEX (PLEXIGLASS) or glass plates held in a frame having a space therebetween; the space may filled with cyanobacteria or algae suspended in nutrient rich water.

FIGS. 4 through 34 illustrate other exemplary systems and methods for production of biofuels.

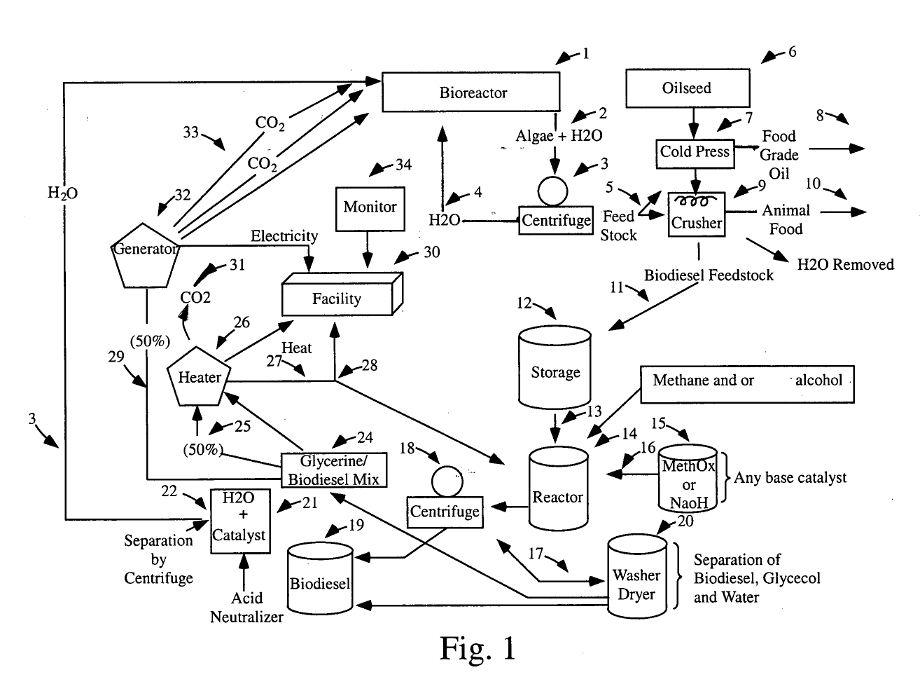

### DISCLOSURE OF THE INVENTION

### Closed Loop

In one particular embodiment the invention provides for a closed loop system that recycles waste byproducts of the biodiesel production process to provide energy to power the process. Other byproducts are used to produce economically useful products such as animal feed, fertilizer and fuel. An important feature of the invention is that it is carbon-neutral, or substantially carbon-neutral. The processes disclosed also require low maintenance and can be run using low-cost substrates.

In one particular embodiment, the “closed loop” system, a suitable biomass may be used as feedstock for the biodiesel production reaction. Such a biomass can be for example, a micro-organism, such as, but not limited to, a blue-green alga, a cyanobacterium, a green alga, Chlorella, green sulphur bacteria, green non-sulphur bacteria, Euglena, a diatom, Cyclotella cryptica, micromonads, and the like. In addition, the invention is drawn to using a bioengineered cell, for example a biological cell, such as, for example, a bacterium, an archaea, or a eukaryote, wherein the biological cell comprises at least one photosynthetic organelle or photosynthetic biological structure. Such organelles can be for example, but not limited to plastids, or chloroplasts, or the like. Such photosynthetic biological structures can be for example, but not limited to, a thylakoid, a photosystem comprising at least one molecule selected from the group consisting of chlorophyll, light harvesting complexes, electron acceptors, pigment molecules, electron transport chain molecules, fluorescent molecules, and the like. In one alternative embodiment, the micro-organism may be adapted for growth in low-light conditions and/or may undergo greater synthesis of lipid, thereby increasing the lipid content of the product.

Alternatively rape (Canola) oil, waste food oil or other oils may be used as a suitable biomass. In another embodiment, other hydrocarbon sources may be used so long as they contain the triglycerides required for the biodiesel production reaction.

In the embodiment wherein algal biomass is used as feedstock, the algal biomass may be produced from the novel “Symbiotic Digestor and Photobioreactor”, also disclosed in this publication.

The closed loop system uses a traditional chemical process to produce biodiesel from a triglyceride and methanol reacted together in the presence of a basic catalyst (such as NaOH).

In one preferred embodiment the system uses a closed loop that is self powered using the biofuels product and/or glycerin by-products of the reaction. All energy-consuming components of the system may be powered by the biofuel/glycerin generators, and all heating and pre-heating functions may be powered by a biodiesel/glycerin heat exchanger. The off-gasses may be used to further feed bioreaction in organisms that utilise carbon dioxide.

The reaction generally employs simple hydrocarbon chain molecules wherein any byproducts are used for secondary production of additional biofuel. The two main components of the present biofuel generator are (1) a closed loop system and (2) a waste digester comprising a bioreactor that maintains two organisms in a synthetic symbiotic relationship.

Novel and useful aspects of the close loop system include the following:

1. It is completely self contained and does not require outside energy input from external utilities to power or heat the plant.

2. It can be containerized because it is self powered. It needs only original feedstock (oilseed rape or algae) and water.

3. In the oil extraction step, there is no hexane extraction required to extract the oil.

4. The seeds are “rough crushed” resulting in a lower content of free fatty acid content of the biodiesel feedstock and also results in lower phosphorous content both of which are desirable to meet international standards for biodiesel.

5. An electrostatic precipitation and/or a negative ion generator is used with the crusher to reduce the undesirable odors. The electrostatic precipitation may also be used for yeast elimination. Other sterilization technologies may be used, such as for example, UHT treatment, or Pastuerization.

6. The glycerine by-product of the reaction is used to heat the facility and/or to provide electrical power, and is generally burned in a hex heater. In some embodiments it is mixed with biodiesel before burning.

7. The biodiesel feedstock may be produced using algal biomass produced from the Symbiotic Digestor and Photobioreactor process. In this case, any carbon dioxide produced is recycled through the growing algal biomass to reduce carbon footprint.

8. Animal food by-product is produced by the system that may be may be charcoaled and sequestered. The consequence is that you produce carbon negative fuel.

9. The closed loop system and the bioreactor may use fresh water or sea water to grow the algae and bacteria, therefore fresh water is not required for the system.

The Symbiotic Digestor and Photobioreactor also disclosed herein may be used independently as a stand-alone bioreactor to produce algal biomass or may be used in conjunction with the “Closed Loop” system disclosed herein.

In the closed loop system, biomass in the form of harvested algae (1), such as from a bioreactor or an algal pond, are fed into a separator and/or centrifuge (3) to separate water from the solid biomass to produce feedstock (5). The solid biomass is fed into a crusher (9). Following crushing it may be mixed with other biomass such as oils which may be produced from rapeseed or other plant matter (6). This product is the biodiesel feedstock. The biodiesel feedstock may be transferred to a storage tank (12) and then fed into a reactor vessel (14). The reactor vessel additionally receives input of methanol (or another alcohol) and a basic catalyst such as NaOH or MethOx (methoxide). Methoxide is an organic salt with a formula of CH3O⁻ and is the conjugate base of methanol. Sodium methoxide, also referred to as sodium methylate, is a white powder when pure and in the present embodiment may be used as a catalyst in the biodiesel reaction.

The products of the reaction in the reactor vessel are biodiesel, glycerol, and water containing basic catalyst. This mixture is now separated to recover the biodiesel. Separation may be done using a simple washer and dryer combination (20) in which the biodiesel is recovered and the glycerol and water are removed, and then separated, one from the other. The glycerol is burned in the hex heater (26). The water may be pH balanced by addition of an acid or buffer solution, and recycled into the bioreactor (1). Alternatively, the reaction product mixture may be fed into a centrifuge (18) that separates the glycerol, water and biodiesel. The water, contaminated by the base catalyst is mixed with an acid neutralizer (22) and fed back into the bioreactor (1). The biodiesel is stored in a storage container (19). The glycerol is burned in the hex heater (26). Tin some embodiments, the solid products of centrifugation are mixed with water and a catalyst and optionally with a glycerin/biodiesel mixture and then fed into a bioreactor (1). The heater and generator (32) are both powered by the burning of glycerol and/or biodiesel, making the whole closed-loop system self-contained.

The system is self-powered using biofuels and/or glycerine by-products. All the integrated biological components, such as the different species of microbes, can be powered by the biofuel/glycerine generators. All heating and pre-heating functions can be powered by a biodiesel/glycerine heat exchanger. The gaseous by-products can, in turn, be further used to feed a bioreaction in organisms that can utilize carbon dioxide, sulfur dioxide, or the like. The system can comprise all the nutrients and micronutrients, the salts, buffers, minerals, and an internal atmosphere, that may be necessary for continuous operation of the closed loop bioreactor.

In one exemplary embodiment, the system includes a bioreactor (1) that comprises a photosynthetic organism having an oil component that is useful for the production of biodiesel. The bioreactor may use fresh water or sea water to grow the algae. This provides an additional benefit in that the systems require no fresh water. A first outlet (2) placed in a suitable position on the bioreactor can allow for constant harvest of the biofuel feedstock. An in-line first centrifuge (3) may is used to separate aqueous media from the biofuel feedstock. A second outlet (4) of the centrifuge may direct the separated aqueous phase (for example, water) from the biofuel feedstock back into the bioreactor; simultaneously, the biofuel feedstock is conducted though a pipe (5) to a cold press (7) that extracts a small quantity of food grade oil (for human or animal consumption) or the biofuel feedstock may be conducted directly to a crusher (9), and from thence though a pipe (8) to a store to be used as food grade oil.

The feedstock for the reactor may be derived from plant seeds having a high oil content, such as, but not limited to, canola, maize, safflower, sunflower, or the like. The seeds can be cold-pressed initially (7) to extract a small proportion of the oils suitable for human consumption.

The composition of oils extracted during the cold pressing may predominantly comprise free fatty acids (FFAs) that are desirable for food products but undesirable for biodiesel production. The remaining feedstock is then conducted to the crusher (9).

The in-line feedstock crusher (9) may have a pre-heating element (NN2) that decreases the viscosity of the feedstock oil prior to crushing. This can help to increase the net amount of oil extracted from the seeds and also aid to increase throughput through a more rapid velocity of the fluid flow. The crusher (9) can be a screw extruder, a press or the like, and can coarsely crush the feedstock (rough crushing). This is preferable to fine-crushing as it leaves sufficiently elevated levels of oils in the by-product that can be used as an animal feed additive (10). The rough crushing also tends to leave the FFAs and phosphorous and related compounds in the animal feed fraction. The animal feedd by-product may be charcoaled and sequestered if desired.

The crusher may also comprise an electrostatic precipitator or negative ion generator, which in use, will cause odiferous compounds, such as thiol-containing organic compounds, hydrogen sulphides, and the like, to be precipitated or removed from the gas.

The oil from the crusher is transported via a pipe (11) to an oils storage tank (12) that may have a pre-heater to increase the temperature of the oil prior to refining.

The stored oil can then be transported through a pipe (16) to the bioreactor (14) that is heated using a glycerine and/or a biofuel heater. A methoxide mixture (15) is conducted through pipe (16) to the bioreactor (14) wherein the conditions in the bioreactor (14) are sufficient to catalyze a chemical reaction whereby the covalent bond(s) between the glycerine moiety and the fatty acid chains, thereby synthesizing a biodiesel. An outlet (17) placed on the reactor can direct the biodiesel to a second centrifuge (18) wherein the glycerine and aqueous phase are separated. In the alternative, the outlet (17) can direct the biodiesel to a diesel washing and/or drying unit (20). The newly synthesized biodiesel can then be stored in a storage container (19).

Water, other aqueous media, and any remaining catalyst are conducted from the second centrifuge (18) or the wash/dry unit (2) through a pipe (21) to a chamber (22) wherein an acid neutralizer, such as a weak base or a buffer, and additional biostock feed that may be used by the photosynthetic organism. In one alternative, the acid neutralizer may comprise nutrients for the photosynthetic organism and can be conducted through a pipe (23) to the bioreactor (14).

Glycerine by-products may be mixed with biodiesel (24) to act as a fuel for a facility heater (26) and/or an electrical generator (32) through pipes or conduits (27 and 29).

The carbon dioxide or other gaseous by-products produced by combustion of the glycerine and/or biodiesel can be conducted through pipes (31 and 33) to an aeration chamber in, or adjacent to, the bioreactor. The carbon dioxide or other gaseous by-products can aid the growth process of the organism in the bioreactor.

In an alternative embodiment, a structure (30) can house some or all of the equipment, storage units, and chambers. The structure can be located in any location, such as on land, at sea, suspended from a balloon, and can also be used in an extraterrestrial environment, such as in a space station, in a satellite, where it can act as a self-contained biosphere, or on the surface of an extraterrestrial body, such as the Moon or Mars, in an bio-equilibrated integrated colony. The system can comprise the necessary facilities, conveniences, and safety equipment for staff and maintenance crews. If the system is at sea or in space, it may further comprise evacuation equipment. The system can also comprise equipment or means (34) used to monitor and regulate ambient and reaction temperature, flow rates of the fluids, chemical properties of the various raw and finished products, and the levels of supplies of substrates, nutrients, and the like.

Algae require about 4 kg of CO₂ to produce 1 kg of algal mass. It is therefore anticipated that the algae or micro-organisms may have growth rates of at least about 50 g algae/square meter/day (g/m²/d). For example, the algae or micro-organisms can have growth rates of between about 50 g/m²/d, or about 55 g/m²/d, or about 60 g/m²/d, or about 65 g/m²/d, or about 70 g/m²/d, or about 75 g/m²/d, or about 80 g/m²/d, or about 85 g/m²/d, or about 90 g/m²/d, or about 95 g/m²/d, about 100 g/m²/d. The systems and methods using such algae can comprise between 50% and 80% lipid (w/w). In one embodiment the lipid can comprise, for example, about 55%, or about 60%, or about 63%, or about 66%, or about 70%, or about 75%, or about 80%.

As noted above, approximately 4 kg of CO₂ is consumed to produce 1 kg algal mass. Under the conditions disclosed herein, 1 kg of algae may produce between 300 ml and 700 ml of biodiesel; for example, 300 ml, 325 ml, 350 ml, 375 ml, 400 ml, 425 ml, 450 ml, 475 ml, 500 ml, 525 ml, 550 ml, 575 ml, 600 ml, 625 ml, 650 ml, 675 ml, and 700 ml, or thereabouts. When the biofuel is burned it will release approximately 3.4 times its original weight as CO₂. Under the conditions disclosed herein the processing of algal mass into biofuel requires a short exposure to ultrasonic waves, less than ½ kWh per 100 m³. Therefore the process may be carbon-negative. Further processing and even amortisation of the carbon construction costs of the materials and equipment still result in carbon negative equations.

The following assumptions can be made: if we assume 540 ml algae are produced the following carbon balance is obtained (0.6 l times spec gravity of 0.9):

4 kg CO₂ consumed

1 kg algae produced

algae processed into 540 ml biofuel

540 ml releases 1.83 kg CO₂

−4kg+1.83kg=−2.17kg CO₂

Therefore subtracting the carbon footprint on building the system and the processing and growth energy costs (for example the pump and aerator operation) and the result is still carbon negative.

### Efficiency of Closed Loop: Requires Little or No Outside Input

One aspect of the system is that it can be designed to reduce the need for outside resources. It is widely known that a closed system algaculture system will be low in need for outside resources, therefore the methods and systems disclosed herein can be effectively self-contained and require only an energy source, such as the Sun, heat from a power generating plant, heat from a home or office building, geothermal heat, kinetic heat, or the like. In some embodiments, the system and the energy source may be small whereby a small closed-loop system is activated by a small, intermittent source of energy, such as an incandescent bulb or a fluorescent bulb. In another example, the debris that is left over after harvesting is quite high in nutrients and minerals necessary for algal growth; this can be recycled as nutrients for the growth of additional algae. For example, if a toilet or a chicken shed is set next to the facility, it may have sufficient nutrients for growth; for example, a chicken shed with 10 chickens would provide enough nutrients for approximately 1000-2000 square meters of panels. A toilet's waste products may perform likewise.

The water in the system can be re-cycled. There is some minor water loss in the finished product, but this is minimal. In the case of using effluent from the distilleries, this is not an issue. The effluent water will be sufficient to keep the system topped up. When attached to a distillery, there is no need for additional input other than waste products from the facility. The effluent is sufficient to provide the necessary elements for growth.

Another aspect of this claim to efficiency is that the byproducts, such as glycerine or post-harvest debris can be burned to generate energy for the facility.

The system can effectively be air-dropped into remote areas to allow for fast deployment and production of biofuels. This will become more apparent later in this document, but, for example, a semi truck container can easily hold 2000 square meters of panels and the processing equipment. This should give 300 litres of biofuel and 100 kg of food per day. NGOs would benefit from this system.

The system design (panels) allow for more than one species of algae being grown. For example, Scenedesmus dimorphus can be grown for fuel while chlorella can be grown as a food (high in protein and omegas). All the resources that may be needed to feed and fuel a community without outside resources can be present within the system or different combinations of the system.

It is also noted that the distillery tests disclosed below have shown that water contaminated with phosphates, nitrogen and even heavy metals may be used. The algae consumes the nitrates and phosphates and “locks” heavy metals. Therefore, the system input is nothing other than contaminated water and CO₂ and the output is oxygen, lipids and protein, thereby equivalent to a “town in a box”.

### Closed Loop can be Fully Automated

All of the control systems for the next stage of development are designed to be remotely operated. For example, the flow rates, water pressures and energy consumption can be easily monitored via data cables.

One example of this is that when the algae is taken into the harvest tank, the ultrasonic system will cause the algae to separate and the lipids will float to the top of the tank. A mechanical arm can float on the top of the oil and allow the oil to be mechanically spill off into a separate tank (see FIG. 23).

Another example is that one species of algae Scenedesmus dimorphus is particularly high in lipid content. The problem is that it tends to clump and drop to the bottom of the reactor (bioflocculation). In conventional tubular systems, this is a problem and requires lots of aeration to break up the clumps. Scenedesmus works well with the panels disclosed herein. In this system, this clumping of algae is an advantage in that the harvesting comes from draining water off the bottom of the reactors. This results in a higher overall algal density in the water that is taken into the ultrasonic harvester thus reducing processing costs. This also increases the ratio of mature (higher lipid) to immature algae being harvested.

The algae does not need constant agitation and CO₂ during the night time. An important novel approach is to allow the algae to settle at the tank during the night-time and harvest first thing in the morning. It has been found that the heavier algae clumps are easily harvested from the bottom of the tanks in the morning. This will also reduce the power requirements and consequently improve the efficiency of the system.

Ii has been found that by creating smaller systems (less than one hectare) there is an exponential decrease in the cost of the infrastructure, equipment and operations.

The prior art is drawn to capturing flue gas from coal burning plants. This has huge costs associated with getting the flue gas into the reactors, and the flue gas from coal is laden with other sulphites and contaminants that hinder growth. Further, if the systems are small it is easier to keep them warm. Unexpectedly, the algae growth rates seem to be more affected by heat than light. Most algae growth is efficient in indirect sunlight at around 10-20% and means that the ponds or reactors used by others requires some sort of shading and consequently cost. The shading in our system can be overcome with the layout of the panels as disclosed below.

### Constant Harvest System

The system is designed to allow for some of the fluid from the reactors to be drawn into a vessel. Within the vessel there can be an ultrasonic probe (56) which breaks the cell wall of the algae and allows the lipids to float to the top of the tank (see FIG. 23) The cell structure which will be composed of proteins and carbohydrates will have the tendency to drop to the bottom of the tank. This material can then be pumped from the bottom of the tank and used as animal feed. This type of processing is considerably less energy and labour intensive than conventional systems.

### Efficiency of the System

One benefit of the invention is that the methods and system disclosed herein are superior in efficiency compared with existing systems.

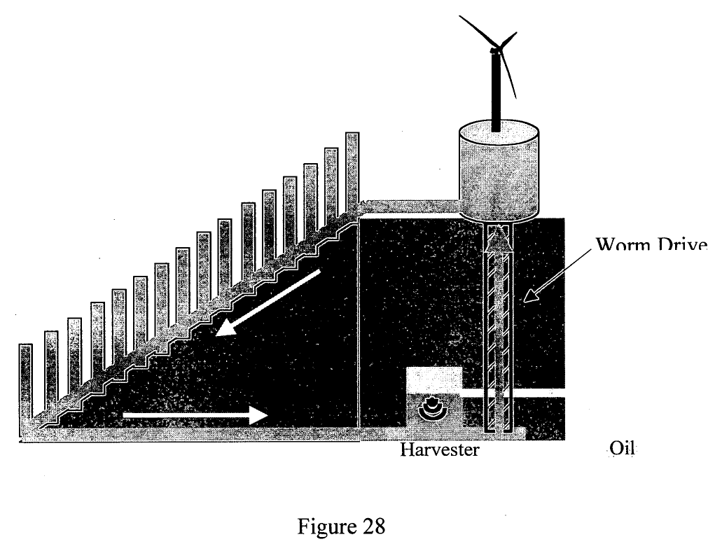

Existing closed systems tend to have tubular plastic bioreactor arrays which each hold vast quantities of water. The volume of the water requires considerable energy input relative to the energy output of the system resulting in a carbon balance that it less favourable than the systems disclosed herein. When incorporating the described conveyer/gravity fed system, the carbon balance is even better (FIGS. 27 and 28).

Existing systems require large pump systems. The described system can be used with a worm drive which recycles the water from the post-harvest reservoir to the gravity tank (FIGS. 27 and 28). There is the additional advantage on a work drive in that it has less damaging affect on the immature algae that are being re-cycled into the panels.

### Semi-Permeable Membrane Reduces Water to Algae Ratio Thereby Reducing Energy Requirements in Processing

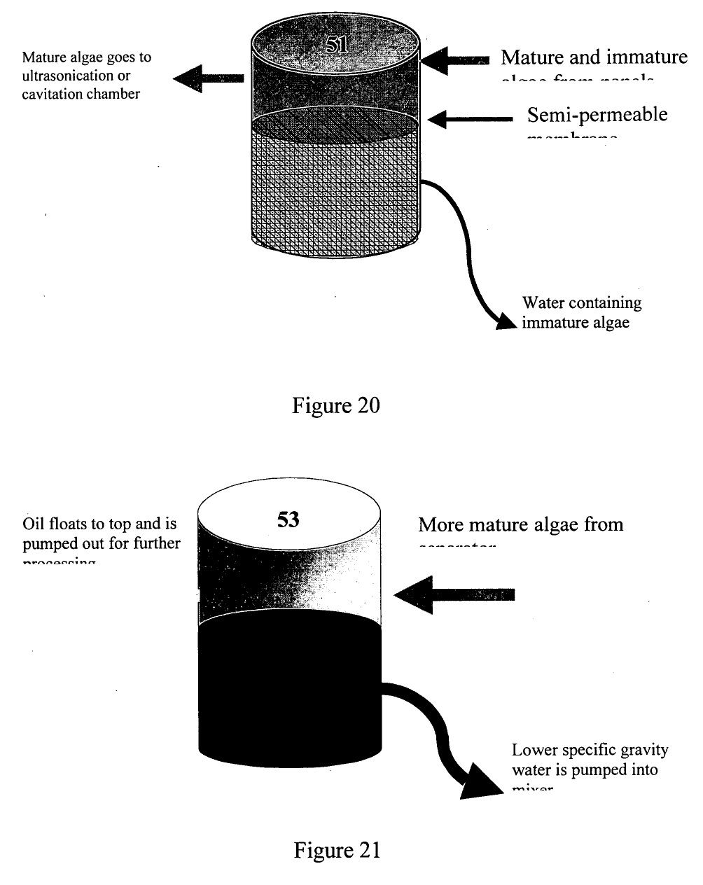

FIG. 20 discloses taking the algae and water slurry from the reactors and allowing the less mature algae to recycle back into the panels before the more mature algae slurry is pumped into the ultrasonic harvesting system in FIG. 23.

The System has a Longer Lifespan than Existing Systems Resulting in a More Favourable Carbon Lifecycle

Another factor in determining the carbon footprint of the system is related to lifespan of components. It is easily argued that a system made from glass has a longer lifespan than that of plastic. The plastic bioreactors that were used became abraded quickly. On a windy day the plastic became visibly abraded from dust and leaves in the air. Plastic is also subject to crazing from the sun and can emit chemicals that inhibit the growth of algae. Even the 1 meter square glass panel that distorted and bowed was left out for weeks in a loose frame, swaying and banging around and didn't actually show any signs of damage.

The lifespan of the panels disclosed herein is estimated to be greater than 10 years, compared with plastic wherein it is harder to argue a lifespan of over 4 years. In addition, at current market prices, plastic costs 4 times more than glass on the instant panel system. In the tubular systems, this cost per meter is even higher.

The previous formula on carbon negativity of the system may also be used for arguing that the system has higher efficiencies than existing systems.

In relation to the energy efficiency we can use the following example,
1 litre of biodiesel has approx 31,976 BTUs or 9.3 kW. If used in heating, you will get upwards of 90% efficiency, if it is used to power a generator there will be about 45% efficiency.
35 panels will produce 1 litre per day assuming 50 g/m2/day with a lipid content of 60% currently each panel requires approx 4 watts per hour for pumps and aerators for 12 hours per day (35×4×12=1.68 kWh for 1 litre)
Processing requires 0.5 kWh thus:
Input is 2.18 kWh, output is 4.18 kWh (9.3 kWh×45% efficiency) for powering a generator; output is therefore 1.92 times the input.
Input 2.18 kWh−output 8.3 kWh (9.3 kWh×90% efficiency) for a heating system; output is therefore 3.81 times the input.
   These are calculations have been run based on 1 litre of algae oil. Using these calculations we note that the energy output is at least about 2.5 kWh per litre of biofuel. These calculations also indicate that the net energy output is at least 0.5 times the energy input or the equivalent thereof. These calculation further indicate that the energy output is at least 1.5 times the energy input or the equivalent thereof with a conversion efficiency of only 35% (generator or heating). For example, the energy output can be 2 times the energy input, it can be 2.5 times the energy input, it can be 3 times the energy input, it can be 3.5 times the energy input, it can be 3.8 times the energy input, it can 4 times the energy input, or it can be greater.

### The “Symbiotic Digestor and Photobioreactor” System

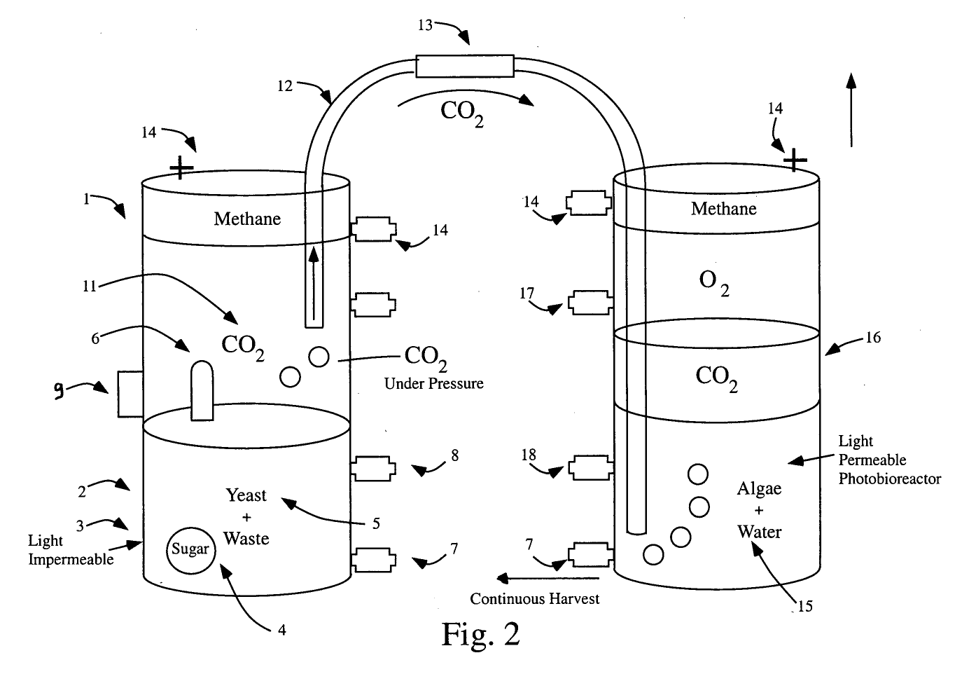

The Symbiotic Digestor and Photobioreactor system uses organisms that use solar energy (algae) to produce covalent bonds between simple organic compounds (carbon dioxide). The Symbiotic Digestor and Photobioreactor is a 2-part closed system. One half of the system (the “left hand side”—see FIG. 2) uses yeast and a carbohydrate source to generate carbon dioxide gas. The other half of the system (the “right hand side”, FIG. 2) uses the carbon dioxide produced to provide a carbon source for the growing algal biomass. Any byproducts generated are re-used in the system.

The input needed on the left hand side is yeast plus a hydrocarbon source, such as biowaste slurry, sugar beat, sugar cane, cellulose material, or any plant or farming by-product. The input required on the right hand side is carbon dioxide (produced by the left hand side) and light.

The product from the left hand side includes ethanol which can be used as a fuel, and hydrocarbon sludge that may be used as fertilizer.

The product from the right hand side is algal biomass which may be used for biodiesel feedstock in the Closed Loop system disclosed herein.

Both sides produce methane, with more methane being produced on the left hand side of the bioreactor.

The invention provides systems and methods for the continuous production of biofuel from biomass. The biomass may be algal, or may be derived from any other source such as lignified or non-lignified plants or the extracts of plants or seeds, such as oils.

In a preferred embodiment the “Symbiotic Digestor and Photobioreactor” system comprises a waste digester and a photo-bioreactor that, in a symbiotic manner, can produce alcohols, such as ethanol, and a biodiesel feedstock, respectively. Carbon dioxide or other gaseous by-products, released during the waste digestion is conducted to the photobioreactor wherein photosynthetic organism(s) incorporate the gas into organic molecules.

The system comprises a (“left hand side”) digestion chamber (1) that can be an opaque plastic, such as polyvinylchloride (PVC) or the like, bag, the bag comprising a material that is inherently impermeable to gases and/or fluids. The digestion chamber can also be manufactured from a metal or a plastic drum, a metal or plastic silo, or any other chamber that is impermeable to gases and/or fluids. In an alternative embodiment, more than one digestion chamber may be used in combination with a bioreactor as described herein.

Note that the terms “right hand side” and “left hand side” are used for convenience to refer to the figure and are not meant to imply any particular positioning of components.

The left hand side chamber is sealed to prevent gases from escaping into the environment and also to allow pressure build up so as to force C0₂ out, into the right hand side photobioreactor.

The left hand side chamber can contain, for example, a slurry comprising a carbohydrate waste (2) and an aqueous medium (3), such as water or a buffered solution of salts. In an alternative embodiment, the left hand side chamber can comprise a gel comprising the carbohydrate waste and aqueous medium and a gelling compound. In another alternative embodiment, the chamber can comprise a porous solid matrix including a carbohydrate waste and aqueous medium.

The left hand side digestion chamber (1) can also comprise a slow-release pellet (4) of waste or sugars or carbohydrates or any other suitable nutrient available to the microbe. In a preferred embodiment the pellet can slowly dissolve over time in order to extend the period during which the digestion occurs.

A micro-organism, such as a yeast (5) is added to the slurry to convert the carbohydrate waste product into ethanol or the like. In one embodiment, the yeast is a naturally-occurring yeast, such as brewer's yeast, Saccharomyces cerevisiae. In an alternative embodiment, the micro-organism comprises a recombinant polynucleotide, wherein expression of the recombinant polynucleotide results in an enhanced rate of reaction for conversion of carbohydrate to ethanol and carbon dioxide. In another alternative embodiment, the micro-organism comprises a recombinant polynucleotide that, when expressed, enables the micro-organism to have a greater tolerance for ethanol and carbon dioxide byproducts. These properties may be important for reaction in a closed system.

The chamber can include a hydrometer (6) that allows monitoring of specific gravity of the liquid in the chamber to allow the ethanol to discharged accurately and at the right time. This system of draining off the ethanol can be automated to maintain the ethanol at an appropriate concentration so as not to kill the yeast. The chamber can further comprise a tap (7) or faucet located on a wall of the chamber that will enable essentially complete drainage of the chamber. The chamber can further comprise a plurality of taps (8) that may also allow drainage of ethanol, resulting from the lower specific gravity of ethanol than water.

The chamber can also comprise an input valve (9) located upon the wall of the chamber that enables controlled addition by a user of micro-organisms, nutrients, water, and the like.

The carbon dioxide or other gas (11) generated during the reaction process creates a positive pressure in the chamber. An efflux valve may be activated by changes in gas pressure can allow the carbon dioxide or other gas to be conducted through a tube (12) and the gas may further pass through a device (13) that can inactivate, immobilize, or filter any contaminating micro-organisms that have been carried with the gas.

The chamber can further comprise a tap (14) located, for example, on the upper wall of the chamber, can allow gases, such as methane, propane, ethane, ethylene, or the like, to be captured or otherwise conducted to another device or system for further use or storage.

The carbon dioxide or other gas then is conducted through a tube (12) to the base of the photobioreactor. The basal region of the photo bioreactor comprises a colony of a suitable micro-organism, such as algae, bacteria, or the like that utilize carbon dioxide to produce biomass. The carbon dioxide or other gas can increase the micro-organism's growth rate. In one embodiment, the carbon dioxide or gas may form a layer (16) at the level of the water and oxygen may be vented through a bypass valve (17) to the exterior of the chamber. Port or valve (17) may also be used to release increased pressure within the chamber and to regulate levels of pressure in the chamber. The pressure can be in the form of a gas. The gas can be methane or hydrogen, or any other energy-rich hydrocarbon. In one preferred embodiment, the micro-organism is tolerant to elevated levels of carbon dioxide or the gas. The micro-organism incorporates the carbon dioxide or other gas into molecules using an endogenous photosynthetic pathway. The micro-organism can be harvested and used to manufacture an oil, a food product, an animal feed, or the like.

In an alternative embodiment, the carbon dioxide gas is provided as a by-product of fermentation from a brewing process. In this case the Symbiotic Digestor and Photobioreactor acts not only to produce useful biomass, but also to sequester carbon dioxide making the brewing process considerably less carbon positive.

Various applications of the Symbiotic Digestor and Photobioreactor process may be employed to sequester carbon in this way and to provide carbon credits to any industry that operates within a carbon trading scheme. The process of sequestering carbon cheaply and effectively makes provides carbon credits and avoids the penalties associated with a net carbon dioxide production.

The micro-organisms may be harvested at predetermined time-points, such as when the cells are near confluence. The micro-organisms may be harvested through a port (18), whereby the water (aqueous phase) and micro-organisms are collected from the chamber, the water and micro-organisms separated from one another using, for example, differential centrifugation, and the water or aqueous phase returned to the photo-bioreactor. In one embodiment, the left hand side or the bioreactor may include bacteria other than yeast. These bacteria help in the decomposition of the waste by-product without concomitant alcohol production. In certain embodiments, the bioreactor may contain an ecosystem that includes amoebae, arthropods, nematodes, mollusks and even crustaceans. In the working model it has been found that snails thrive and consume oxygen while producing CO₂. The organisms within the biosphere create a symbiotic, self-sustaining biosphere that degrades waste and produces carbon dioxide. In some commercial embodiments, it may be that it is more desirable to eliminate these micro-organisms.

The system can be located on the ground or it can be located on water or in any other location as disclosed herein.

Ethanol can be drained as needed to allow the continuous bacterial reaction. As ethanol is drained, the digestor chamber can be topped up with additional biomass and bacteria. Likewise the algae can be harvested in regular intervals thereby keeping the interaction between the digestor and the bioreactor constant. Methane could also be regularly tapped. In addition, the ethanol may be sequestered and used as a biofuel.

A number of Symbiotic Digestor and Photobioreactors may be coupled together so that the product from one feeds another. As illustrated in FIG. 34, one large digestor can supply more than one bioreactor. Ethanol has a specific gravity of 0.79. The ethanol can be drained as needed to allow the continuous bacterial reaction. As ethanol is drained, the digestor chamber can be topped up with additional biomass and bacteria. Likewise the algae can be harvested in regular intervals thereby keeping the interaction between the digestor and the bioreactor constant. Methane may also be regularly tapped.

The systems disclosed herein can work independently or together. The closed loop system can be fed by oil algal biomass or any triglyceride containing substance to make biodiesel. The Symbiotic Digestor and Photobioreactor system can be used to produce biomass from and waste or carbohydrate source that may be microbiologically digested to produce carbon dioxide. The systems can be used together so that the Symbiotic Digestor and Photobioreactor produces algal biomass that acts as feedstock for the closed loop system. The lack of requirement of either system for an external energy source or for fresh water makes the system versatile, inexpensive and portable. Additionally very little maintenance is required. This makes the system ideal for poor economies or for situations in which resources, energy or land is in short supply. Additionally, the closed loop and Symbiotic Digestor and Photobioreactor systems do not require displacement of food crop producing lands for fuel production because non-arable lands can be used; desert areas are well suited to algae growth. The closed loop system is ideal for isolated or rural areas that do not have electricity, limited water supplies. The systems described produce biofuels cheaply and with little external intervention by the user are described. The systems can be produced in small sizes sufficient for a single family home and are particularly useful for use in regions of the Earth where there is continuous sunshine but low availability of fossil fuels.

The invention uses the advantage of natural sunlight energy that is converted by a biological organism (or derivative thereof) into atomic bond energy between two atoms. The bond can be a bond in an organic molecule, and is preferably a covalent bond, but other high energy bonds are included. The biological organism (biomass) uses substrates such as carbon dioxide (CO₂), water, methane, and the like, to synthesize hydrocarbon molecules, such as carbohydrates, lipids, alcohols, aromatic compounds, sterols, and the like, that can be separated from the biomass, purified, and distributed for use as a fuel. In order to enhance productivity, additional nutrients, such as nitrogen, calcium, iron, copper, usually in the form of salts, may be added to the biomass.

The micro-organisms may be harvested at predetermined time-points, such as when the cells are near confluence. The micro-organisms may be harvested through a port, whereby the water (aqueous phase) and micro-organisms are collected from the chamber, the water and micro-organisms separated from one another using, for example, differential centrifugation, and the water or aqueous phase returned to the photobioreactor.

The micro-organisms are tended as a microbial biomass within a reactor chamber, the chamber comprising modular panels of a translucent material that create a sandwich with the microbial biomass. In one embodiment, the invention comprises the modular panels that further comprises a brush and magnet combination (scrubber) that, in use, may be used to periodically clean the inner surface of the plate, thereby allowing maximal photonic energy to be transmitted therethrough as well as increased capacity and throughput of biomass. The brush and magnet combination may be mobilized to traverse the surface of the plate using an opposing magnet positioned upon the exterior surface of the plate. In another alternative embodiment, a plurality of scrubbers can be positioned so as to direct the passage of feeder gas (for example CO₂) through the biomass and the chamber, thereby enabling better absorption of the gas by a greater proportion of the micro-organism and improved growth potential. The reactor can be adapted for positioning to face the Sun (or other light source) at a preferred angle to the ground or surface. A preferred angle may be dependent upon the season and the reactor may be repositioned according to the angle of the incident light. In winter, for example, the reactor may be positioned at an angle that is approximately 22.5° greater than the latitude at which the reactor is located on the surface of the Earth. In summer, for example, the reactor may be positioned at an angle that is approximately 22.5° less than the latitude at which the reactor is located on the surface of the Earth. The reactor panel(s) may also be rotatable about an axis, thereby allowing a panel to be rotated as the Sun traverses the sky so as to provide the panel with maximal photonic energy during periods of daylight.

The reactor chamber is adapted for placement and/or fixedly attached on the surface of any structure, on the surface of the ground, or it can be placed on water or in any other location as disclosed herein. In one embodiment, Kaser (2007) has suggested that electrical energy producers pump gaseous CO₂ released by burning fossil fuels through vast transparent vats filled with blue-green algae and nutrients. The vats would be placed on the roofs or the sides of a building facing the sun, and algae would grow using the sunlight and the excess CO₂ produced by fossil fuel combustion. The algae could be periodically (or continuously) harvested and refined as a biofuel, thus reusing the carbon expelled from the energy plant (Kaser (2007) “The power of pond scum” High Country News (ISSN/0191/5657), Paonia, Colo., USA, Letters, Oct. 15, 2007). The system may sold as a fume scrubber that produces oil and carbon credits. The processed oil may be decanted from the tank and further process into biodiesel. In addition, ethanol may be periodically decanted for use as a fuel additive.

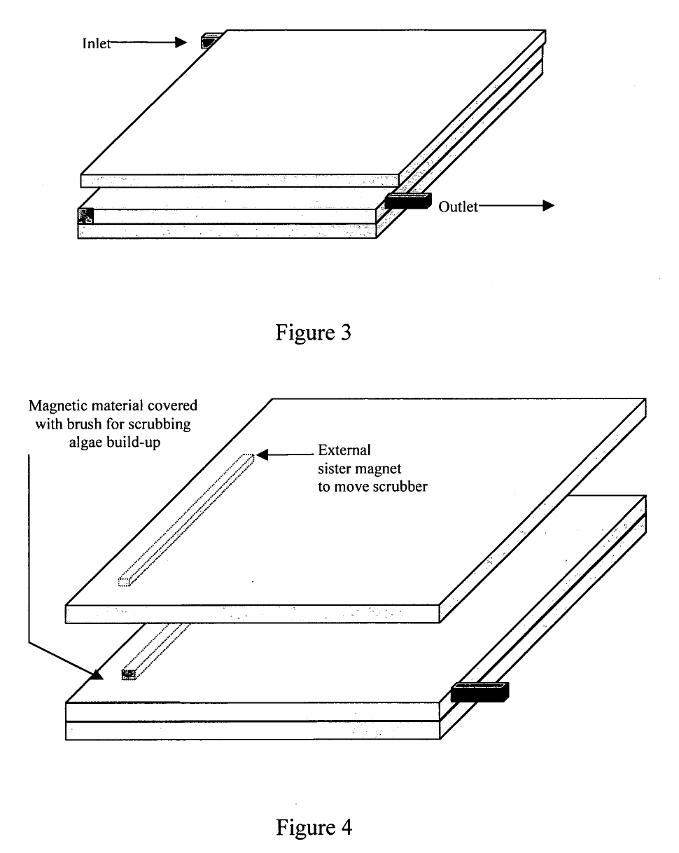

FIGS. 3 through 2X illustrate particular exemplary embodiments of the modular systems (for example, panels) comprising micro-organisms that may be used for the synthesis of biofuel.

FIG. 3 illustrates a unit tile or panel construct comprising two plastic (for example, PERSPEX/PLEXIGLASS or the like) or glass plates held in a frame with a space therebetween of between 5-500 mm. The space can be an airspace having a dimension of about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 110 mm, about 120 mm, about 125, mm, about 130 mm, about 140 mm, about 150 mm, about 160 mm, about 170 mm, about 175 mm, about 180 mm, about 190 mm, about 200 mm, about 210 mm, about 220 mm, about 225 mm, about 230 mm, about 240 mm, about 250 mm, about 260 mm, about 270 mm, about 275 mm, about 280 mm, about 290 mm, about 300 mm, about 310 mm, about 320 mm, about 325 mm, about 330 mm, about 340 mm, about 350 mm, about 360 mm, about 370 mm, about 375 mm, about 380 mm, about 390 mm, about 400 mm, about 410 mm, about 420 mm, about 425 mm, about 430 mm, about 440 mm, about 450 mm, about 460 mm, about 470 mm, about 475 mm, about 480 mm, about 490 mm, and about 500 mm. The airspace may be filled with a micro-organism, such as for example, but not limited to, cyanobacteria or algae, suspended in nutrient rich water. There is an inlet and outlet to allow for constant feeding, re-populating and harvesting of the micro-organism. There is an outlet for harvesting the micro-organism and biofuel. In one example, panels can be connected together. The inlet and outlet may have isolating valves which can allow for the repair or replacement of tiles or panels.

The dimensions of the unit tile or panel can be a rectangle of about 5 cm×50 cm, or about 10 cm×50 cm, or about 10 cm×1 m, or about 10 cm×1.5 m, or about 15 cm×2 m, or similar combination. The unit tile or panel can be a square shape having sides of about 10 cm, about 20 cm, about 25 cm, about 30 cm, about 40 cm, about 50 cm, about 60 cm, about 75 cm, about 100 cm, about 150 cm, or about 200 cm.

FIG. 4 illustrates an additional example of such a tile or panel wherein in each tile there is a brush with a magnetic centre-piece (a “scrubber”) that allows for easy cleaning of built-up algae on inside surface of tile. There may be three of these scrubbers, held in position to allow for greater travel distance. Cleaning of built-up algae improves the efficiency of energy transfer through the panel walls thereby allowing more energy to be available to the micro-organisms.

Alternatively, the inner surfaces of the tile or panel is covered with a membrane that prevents adherence of the micro-organism to the inner surface. Such a membrane can comprise a synthetic material, such as TEFLON, cellulose acetate, polyvinyl chloride, polyurathane, silicone rubber, and the like or it can comprise a biological material, such as, for example, collagen, fibrin, cellulose, lipid-conjugates, and the like.

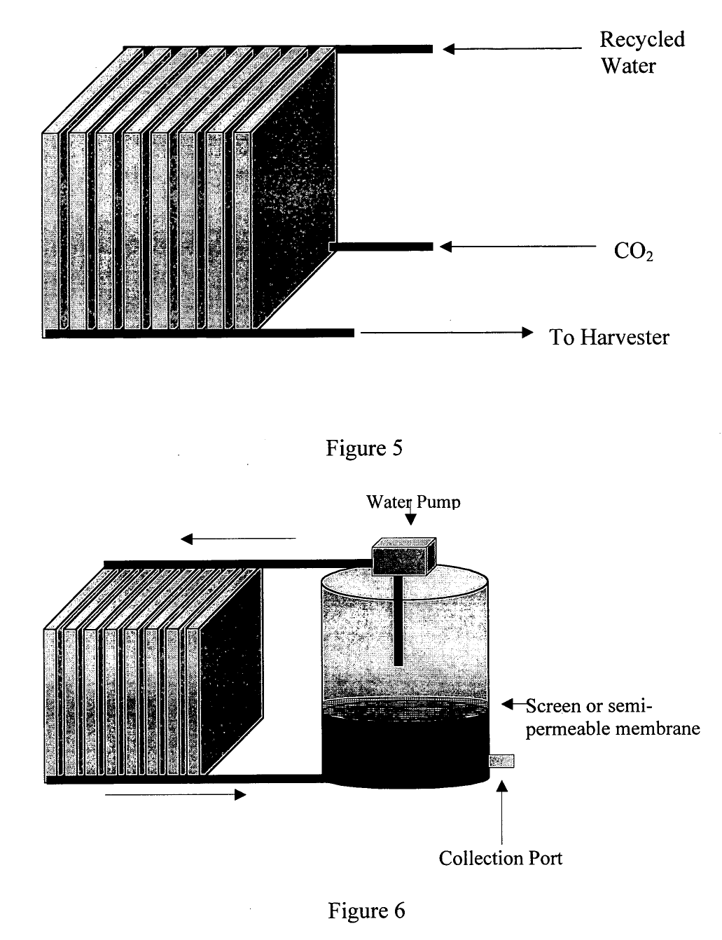

As illustrated in FIG. 5, the panels can be stacked with small air gaps between them to allow sufficient sunlight through whilst remaining compact.

FIG. 6 illustrates a further embodiment of the panel arrangement disclosed in FIG. 5 whereby the panels are linked to a micro-reactor unit comprising a water pump, a screen or semi-permeable membrane, and a collection port, from which oil and other products may be tapped.

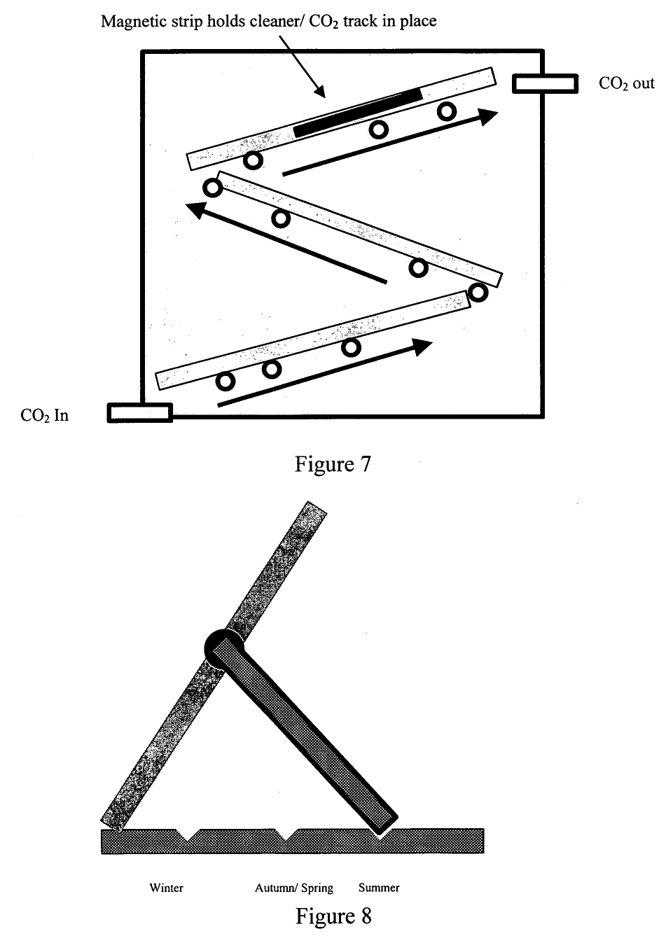

FIG. 7 illustrates one optional variant of the system wherein increasing the travel distance of the CO₂ allows for better absorption and subsequent growth of the algae or micro-organism. The scrubbers disclosed in FIG. 4 can be held in place by magnets. The scrubbers can also act as tracks along which the CO₂ (small bubbles) is guided (arrows) as it rises through the tank or panel.

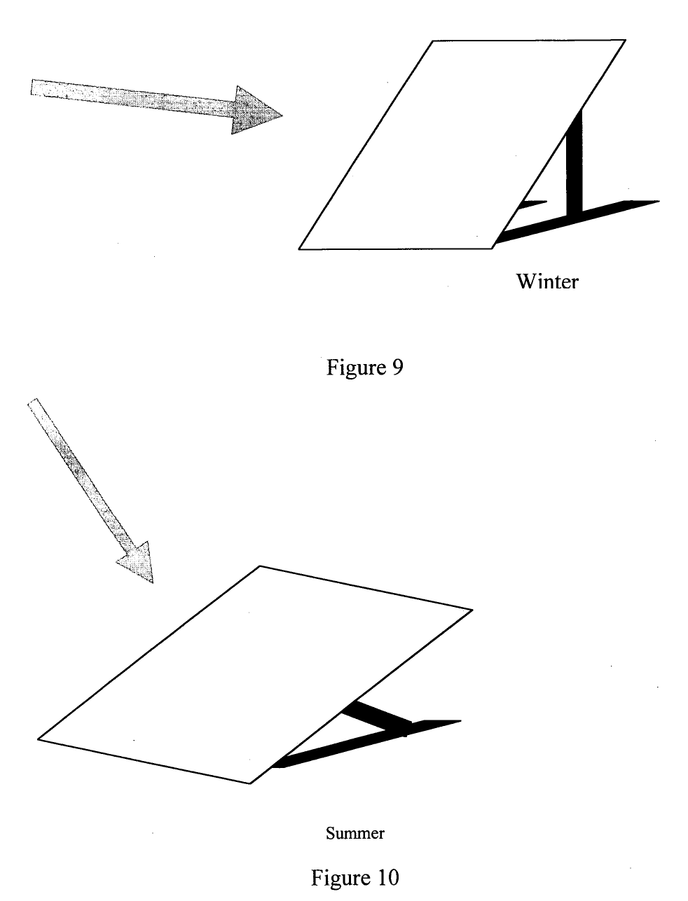

FIGS. 8, 9, and 10 illustrate how the panels can be positioned upon a surface and the angle of the panel may be adjusted to accommodate the direction of the incident light source. If the panels are positioned on a surface having notches therein, they can be easily adjusted to maximise the position of the sun during various seasons. The panel can comprise an adjustable frame for cultivation of micro-organisms and/or algae when the sun is at different azimuth or declination so that the system can be adapted for use anywhere on Earth. In addition, the system can be used in an extraterrestrial environment, such as aboard a spacecraft or anywhere upon the surface of a planet or moon.

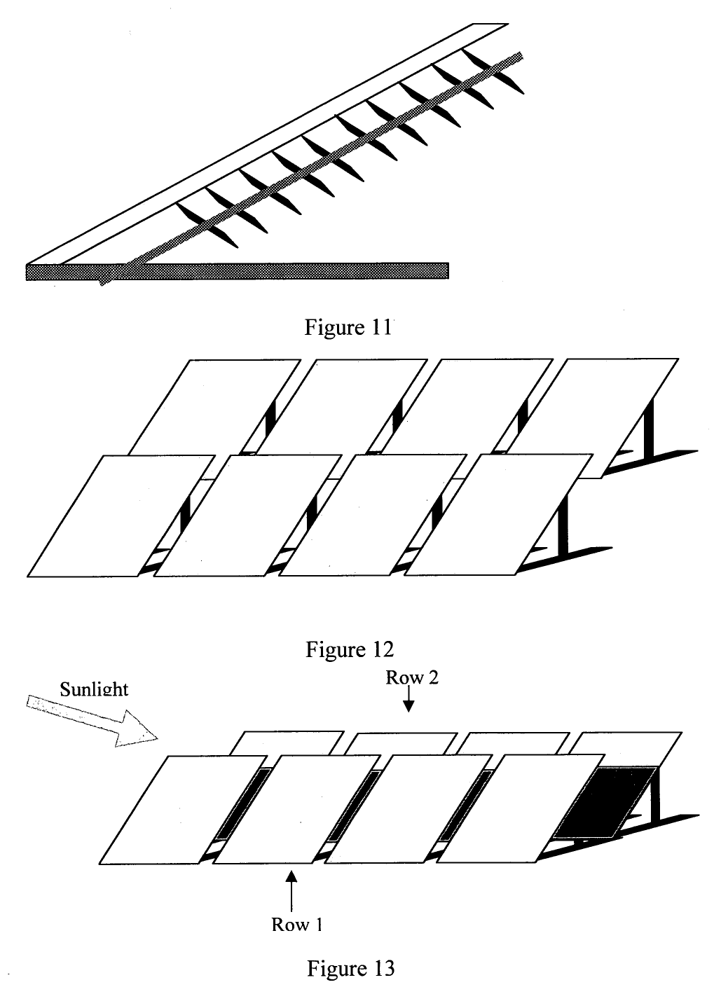

FIG. 11 illustrates another alternative exemplary embodiment, the back of the panel having a system of blinds or louvers that allows for heat absorption or reflection either to the micro-organisms or to a heat sink. The blinds can also be used to regulate light absorption or reflection. One side of the blind can be black to allow absorption of the infra-red energy from the sun when needed. Alternatively, the other side of the blinds may be coated in a reflective material thereby reducing the absorption on hotter days. Likewise, when the panels are used as a building material, the blinds may be used to regulate temperature and increase or reduce heat loss.

FIG. 12 illustrates another embodiment whereby the panels can be ganged together in series or in parallel, thereby allowing many small panels to be combined to create a larger surface area. One advantage is that should one unit be damaged or require service, only a small portion of the system need be removed for servicing, for example, cleaning or maintenance, without needing replacement of a larger system, thereby incurring a potential cost savings.

Some species of algae flourish in the maximum light available whilst some species prefer diffused sunlight. As shown in FIG. 13, row 1 comprises a species which prefers direct light whilst the micro-organisms of row 2 prefers partial shading of light. This also allows for the maximum use of the area of the algae farm in that additional pair of rows, comprising row 3 and row 4 as illustrated in FIG. 15, whereby row 3 may be positioned at a distance to allow for access whilst row 4 will have the benefits of partial shading. Panels can be arranged to allow maximum exposure to the sun.

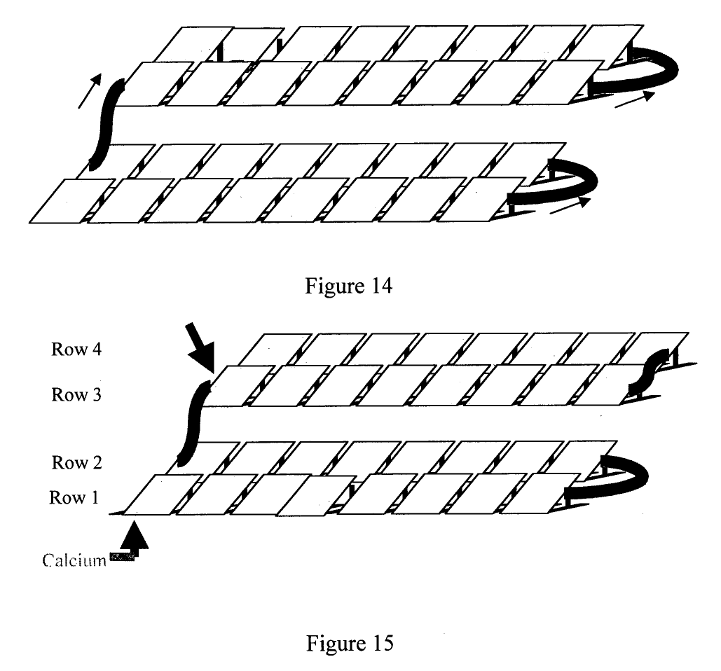

In another exemplary embodiment, the panels may be daisy-chained together allowing for the constant flow of fluids and nutrients through the array of tiles, as illustrated in FIG. 14. Some species of algae only bloom during colder months while others flourish in the summer. The design of the panels allows one to completely drain the panels and re-populate them with season-specific species. Arrows indicate water flow direction.

FIG. 15 further illustrates a system whereby the tiles or panels may be daisy chained together allowing for the constant flow of fluids and nutrients through the array. Algae require different nutrients during the various stages of growth. In this example, the system allows for introduction of calcium for cell wall growth during the early stages of development where lipid producing nitrogen is introduced when the colony is more established. In the example above row 1 receives calcium to stimulate cell wall growth while row 3 receives nitrogen to stimulate lipid production.

In many countries compacted snow is common on rooftops and considerable work has been done to ensure roof structures can take the additional load. Most roofs must be built to withstand weights of over 200 kg per square meter. In that a 1 m² panel of 30 mm depth will have a total liquid capacity of 30 litres (30 kg) and the tempered glass and plastic frame will weigh approximately 40 kg the total weight of the algae and water filled panel will be less than 70 kg which is equal to or less than many existing building materials. In that algae will be grown primarily in areas that are not subject to large amounts of snow, the tiles will be well suited as a building material as roof tiles. In that the preferred material is either plastic or tempered glass, the materials are already CE and or UL marked they are suitable for wall construction. For example, tempered glass is the equivalent of safety glass

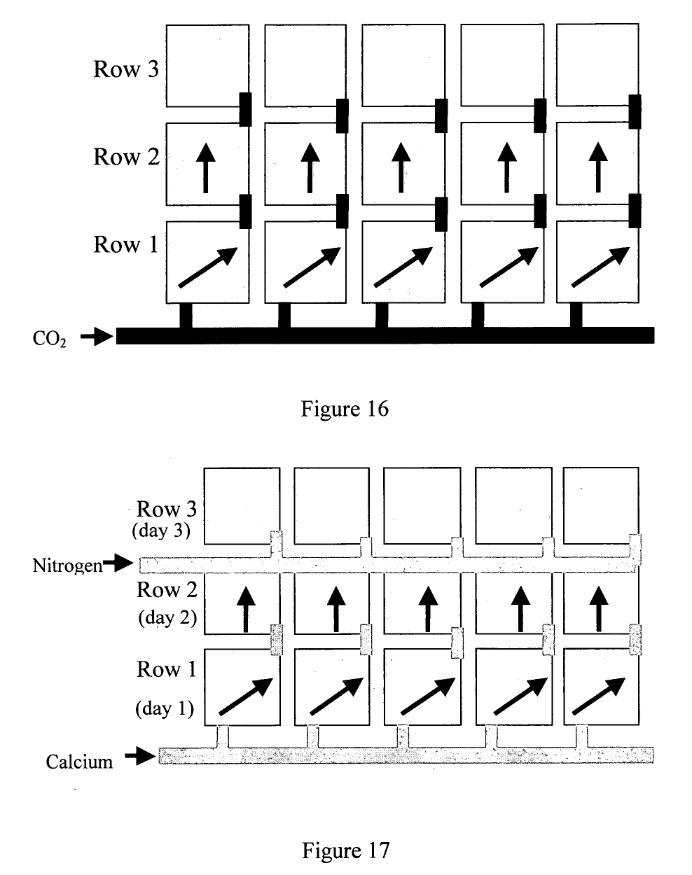

The tiles or panels can be used as a building wall or roof. In this embodiment, as shown in FIG. 16, CO₂ percolates (arrows) through row 1 then rows 2 and 3 allowing for the maximum amount of CO₂ absorption by the algae.

FIG. 17: Nutrients can be added during the life cycle of the algae which can maximise the efficiency of the reactor. Assume that the algae moves from the first panel in row 1 to row 3 during a 3-day maturation cycle. Calcium can be added in row 1 while lipid producing nitrogen, which is preferable for biofuel production can be added on day 2.

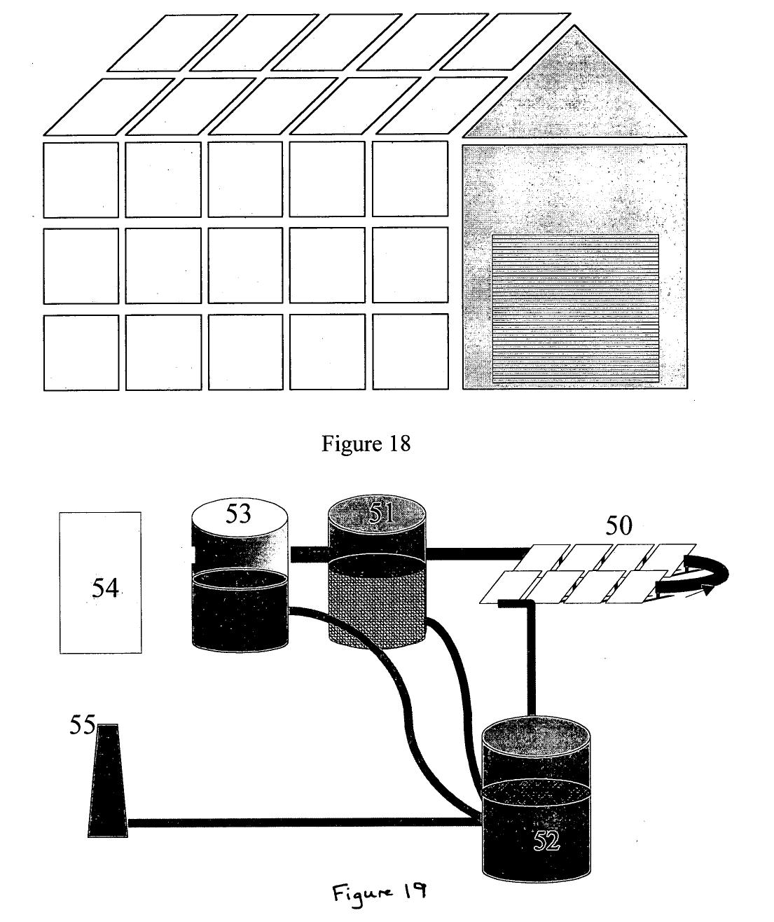

Algae filled panels can be used to construct buildings, as illustrated in FIG. 18. If panels are made with tempered glass, they will generally meet with most EU and US building requirements. In that many of the applications will be using excess heat from industrial processes, for example, distilling and energy generation, the issue of snow and frost build-up will be mitigated by the warm water in the panels.

FIG. 19 illustrates a combination of the two systems disclosed herein.

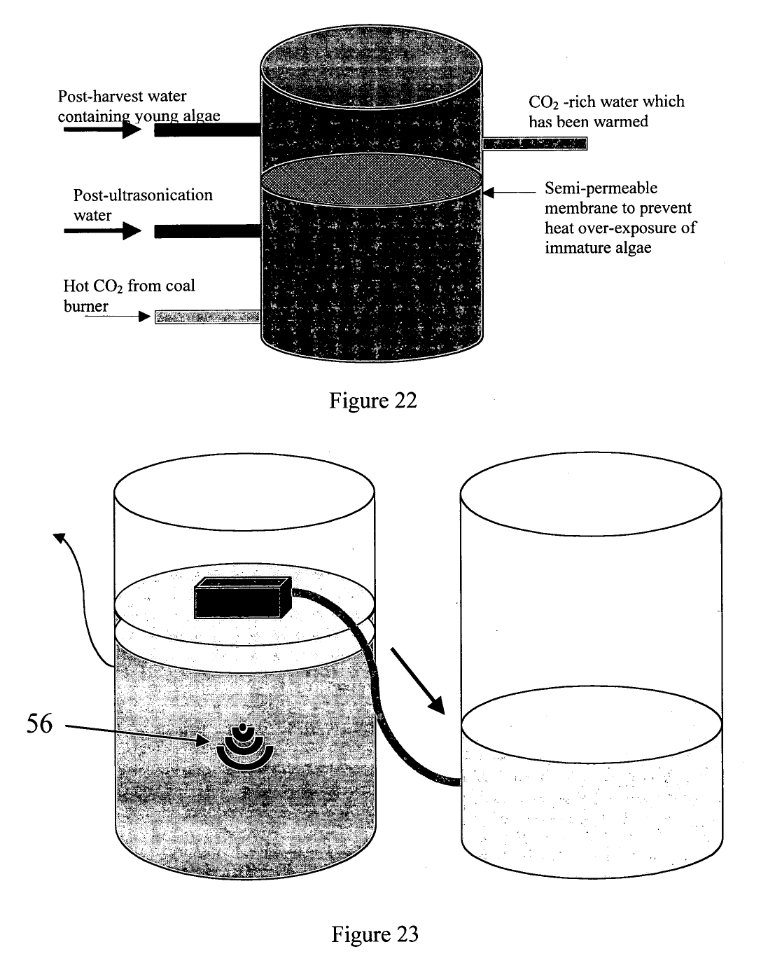

Flue gasses from industrial processes are often in excess of 100° C. At the same time, water often needs to be heated to maximise algae growth rates. In this example, shown in FIG. 19, the water containing both mature and immature algae (50) is pumped into a separator (51) where a semi-permeable membrane allows the less mature algae to be separated and pumped back into the mixing tank (52). The mature algae is then pumped into a tank (53) where ultrasonic and or mechanical cavitation causes the algae cell wall to rupture releasing the lipids which float to the surface of the tank. The lipids are pumped into a reactor (54) for processing into biofuel while the remaining water from the separator (53) is pumped into the mixing tank (52). Flue gasses (55) are piped into mixing tank (52) where it warms the recycled water from tanks 51 and 53. Mixing tank (52) also may contain a semi-permeable membrane to reduce the exposure of the immature algae to high temperatures. More detailed drawings are illustrated in FIG. 20 (detail of separator), FIG. 21 (detail of ultrasonication/cavitation tank), and FIG. 22 (detail of mixing tank).

FIG. 23 illustrates another alternative embodiment wherein a mechanical arm can float on the top of the oil and allow the oil to be mechanically spill off or drain into a separate tank and the water is then recycled back to the reactor.

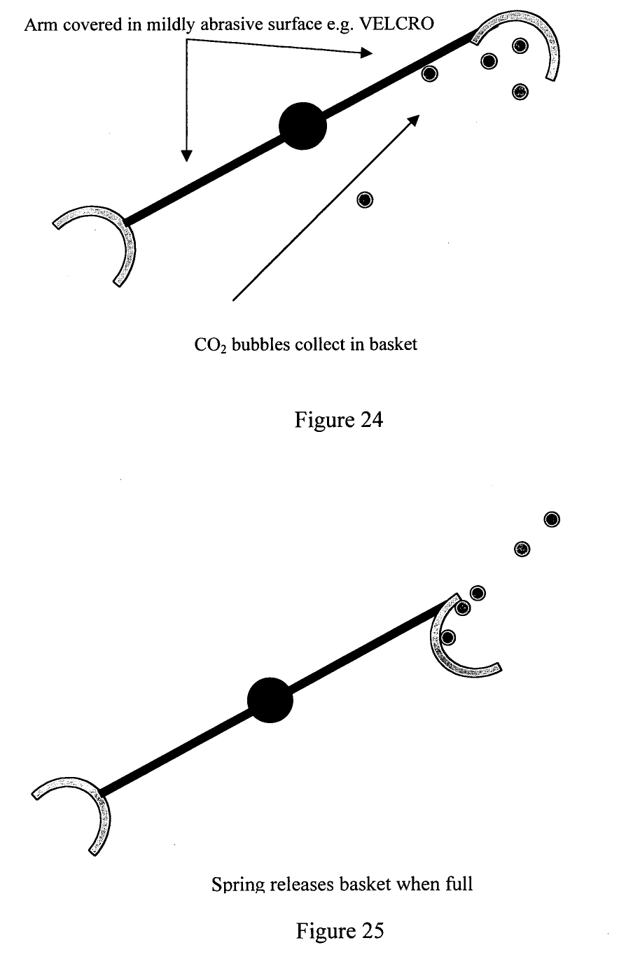

FIGS. 24 through 26 illustrate another embodiment wherein a rotating bracket for collecting CO₂ bubbles is used to keep the algae from collecting on the surface of the glass and thereby decreasing the transmission of solar energy. At the same time it allows the algae to have increased exposure to the carbon dioxide bubbles.

FIG. 27 illustrates how a gravity-fed tank with ultrasonic harvester and enclosed worm drive raises water for use in a series of panels or units that in turn, feed micro-organism crude biofuel products to a second bioreactor wherein the biofuel is harvested and directed to a storage container.

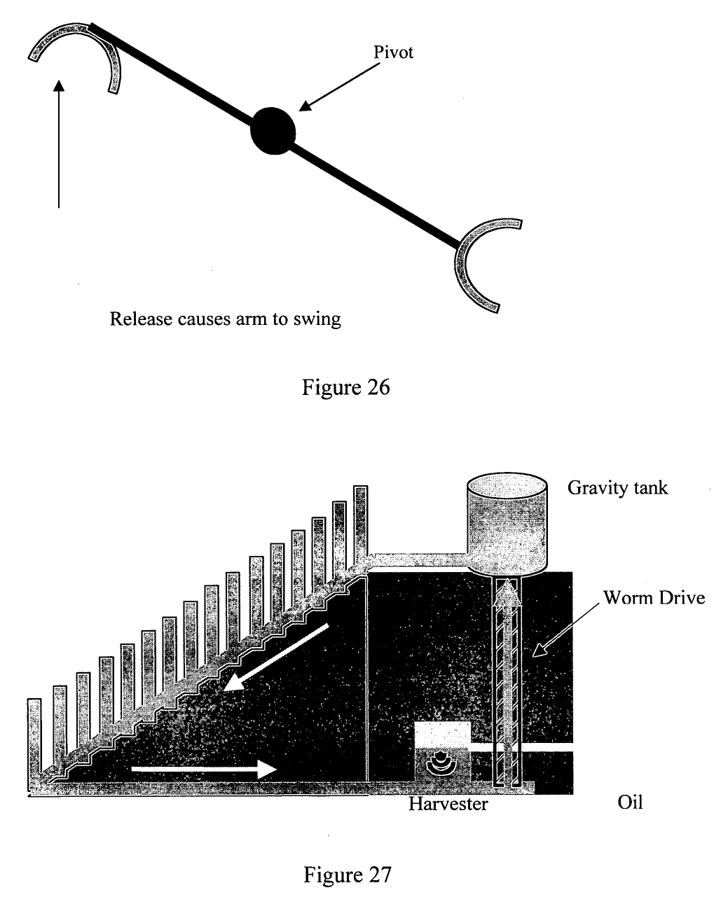

FIG. 28 illustrates an alternative embodiment of the system of FIG. 26 whereby the worm drive is driven by a wind turbine.

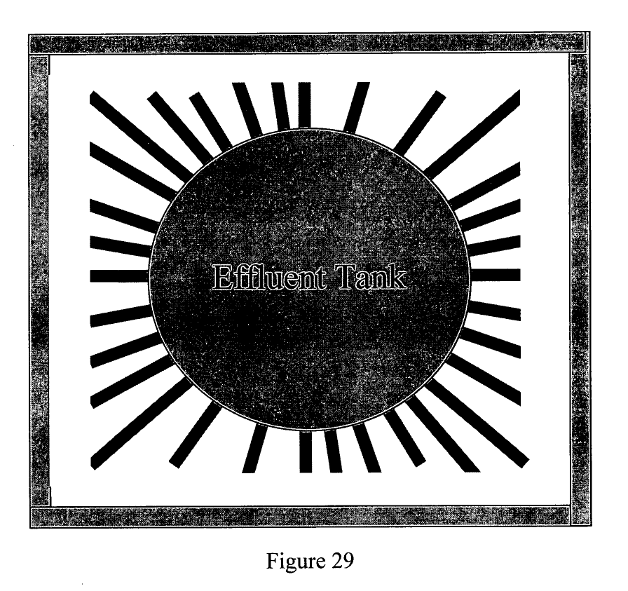

FIG. 29 illustrates an exemplary array of panels placed adjacent of the exterior of an effluent tank.

### Configuration of Panels Allow Vertical Stacking to Increase Density and Yield Per Square Meter

The panel design allows one to position them with small air gaps between the arrays. For example, if the panels are 25 mm thick and are placed 25 mm apart a total of 20 panels can be placed on a 1 meter area. This allows sufficient light to penetrate each panel while keeping the footprint of the array quite small. In the above example you would have an effective algal surface area of 20 square meters. (See FIGS. 5 and 6). This may be important in that a system with an area of 5 cubic meters would have a total of 200 square meters of algal surface. If the system is producing 50 g/m²/day then it would produce 10 kg of algal mass resulting in about 6 litres of biofuel. In one embodiment, these may be used as roof-top reactors.

### Configuration of the Panels Used as Rooftop Microscrubbers and Microrefineries

One cubic meter of panels, positioned on a rooftop of a building would consume 40 kg of carbon dioxide and produce 6 litres of biofuel and 3 kg of animal food per day. This is important for the production of small systems for any business or building that uses oil based heating systems. For example, a business burning 120 litres of oil per day would produce about 400 kg of carbon dioxide. If the scrubber on the roof is cutting CO₂ output from the business by 10% and they are burning the renewable oil they may obtain government credits (such as “renewable certificates”). Such a business could also produce up to about 2,000 litres of oil per year and consume a total of 14 tonnes of CO₂ per year.

Such panels may also be used to flue gas from ships and similar craft, such as cruise liners, ferry boats, oil tankers, container ships, and the like. Smaller sets of panels may be attached to the upper surface of transport vehicles including trucks and trains. A semi truck would be able to produce 10,000 miles worth of biofuel per year and consume 20 tonnes of CO₂. In addition, such panels may be placed upon the upper surface of a standard container, thereby providing additional means for producing biofuel during transportation.

The system may also have an ultrasonic constant harvest system attached to panels whereby the water is cycled through the ultrasonic harvester and the oil is siphoned off into a holding container which can then be used by the operator.

### Stand-Alone Micro-Reactors

In some cases it may be economically beneficial to place small arrays of photobioreactors in areas where CO₂ is generated but not in sufficient quantities to justify a refining element.

For example, a building may have several cubic meters of reactors which consume the CO₂ generated from the heating system or other industrial applications. The first stage of growing and harvesting can involve simple mechanical filtration and automated feeding. The filtration system may allow for periodic collection of the highly concentrated slurry which is then brought to a separate facility for processing. (See FIGS. 5 and 6.) It is anticipated that the capital costs for a 2 cubic meter system is a few hundred dollars or equivalent. Forty panels may be placed in that 2 cubic meters which could convert 8 tonnes of CO₂ into 1 tonne of fuel and 1 tonne of food product per year. Alternatively it may be charcoaled and sequestered.

Carbon Sequestration through Charcoaling

Alternatively the products from the micro-reactor may be charcoaled and sequestered. The charcoal may then be used in industrial processes, such as manufacture of steel or barbeque pellets, or it may be used in a domestic environment as a source of heat for cooking in regions having low density of woodland, for example, the Sahel or regions proximal to major deserts. This also may be used to as a commodity on the carbon markets.

### Separation of Metals and Heavy Metals Post Charcoal

One unexpected result was the capture of copper ions by the micro-organisms, combining such ions in the by-product, and removal of concentrations that might otherwise be toxic from effluent. In general it has been understood that copper will generally kill algae, but certain cyanobacteria are apparently capable of capturing metal ion through bioleaching. For example, Chlorella may bind copper with copper binding proteins, such as, for example, a plastocyanin. In the alternative, it may result from some mechanical function and that the copper may get stuck to the algae or colonies will clump and inadvertently isolate the copper. In one embodiment, therefore, the system and methods disclosed herein may be used to sequester and/or salvage metal ions that contaminate effluent prior to extraction of a biofuel. An additional benefit is that the micro-organisms may be used to detoxify large areas of contaminated soil and vegetation that would otherwise incur considerable costs if other chemical remediation were to be used. Post oil separation, the by-product may still contain such metals, including heavy metals. In these cases there may be an advantage to use some bioleaching or biochelatic process such as disclosed herein compared with those described in the prior art (see, for example, Tam et al. 1998 Biotechnol. Tech. 12(3): 187-190)

### EXAMPLES

The invention will be more readily understood by reference to the following examples, which are included merely for purposes of illustration of certain aspects and embodiments of the present invention and not as limitations.

### Example I

### Implementation of Biofuel Generator System at a Distillery

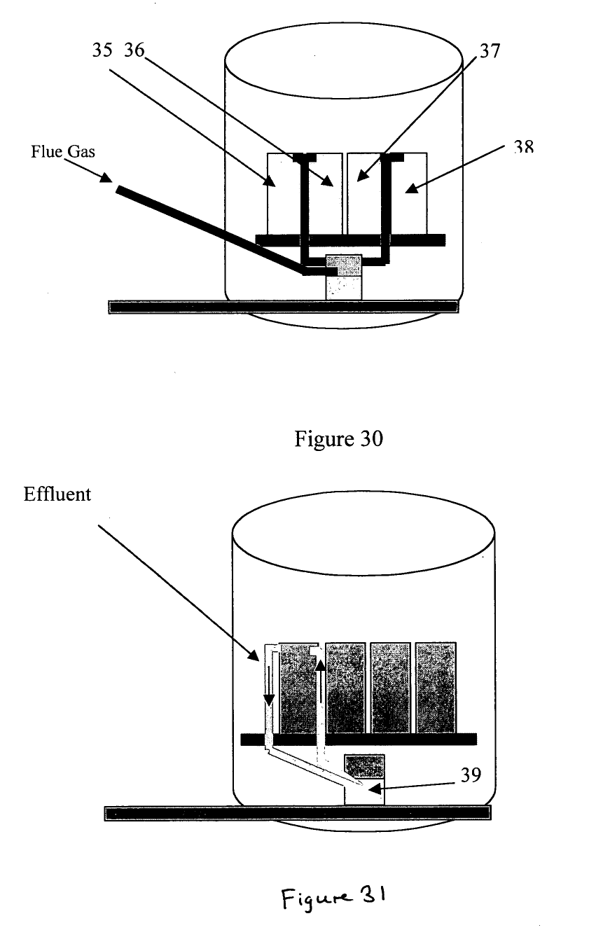

The panel system was tested at a Scottish distillery. Panels were placed on a horizontal frame adjacent to an effluent tank as shown in FIG. 30. The distillery tests revealed several unexpected discoveries.

### Effluent

One of the tests panels was using effluent from the malting and distilling process. This effluent is high in nitrates, phosphates, copper, and copper ions. Currently the distillery pays farmers to collect and dispose of the effluent on their fields. However, recent proposed changes to the national legislation will disallow the disposal of effluent in this manner.

The test comprised the following three experiments to test the effect of flue gas: 1) directing flue gas from the effluent tank gas into a panel (35 & 36) comprising algae (Chlorella vulgaris); 2) control algae, no flue gas (37); and 3) an empty panel (38) into which flue gas was directed. In parallel, as illustrated in FIG. 31, effluent from test panel 35 was recycled back to the panel using a 23 W re-circulating pump (39). Table 1 shows the compositions of each of the four test panels.

TABLE 1

| Panel 1 | Panel 2 | Panel 3 | Panel 4 |
|---|---|---|---|
| 2 litres algae | 2 litres algae | 2 litres algae | No algae |
| 1 litre effluent | 18 l distilled | 18 l distilled | Flue gas only |
| 17 litres distilled | nutrients | nutrients | to determine |
| nutrients | food | food | sulphur, nitrates, |
| food | flue | | etc. transferred |
| flue | gas | | from gas |
| gas | | | |

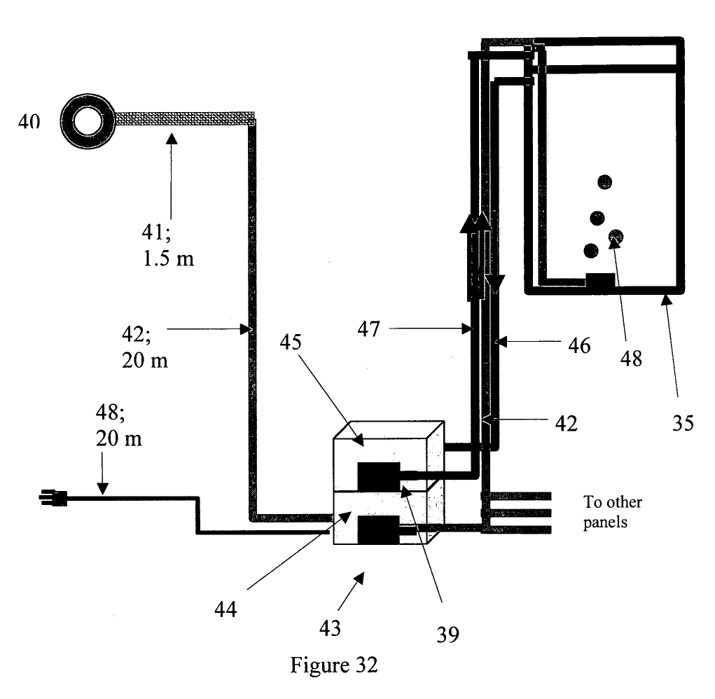

FIG. 32 illustrates in more detail the design setup showing the flue (40), high-temperature hose to collect flue gases (41), flue gas line (42), flue gas pump (43) re-circulating pump (39), nutrient chamber (44), recycling chamber (45), effluent lines (out: 46 and in: 47) and flue gas (48) entering test panel (35), and a power cord (49).

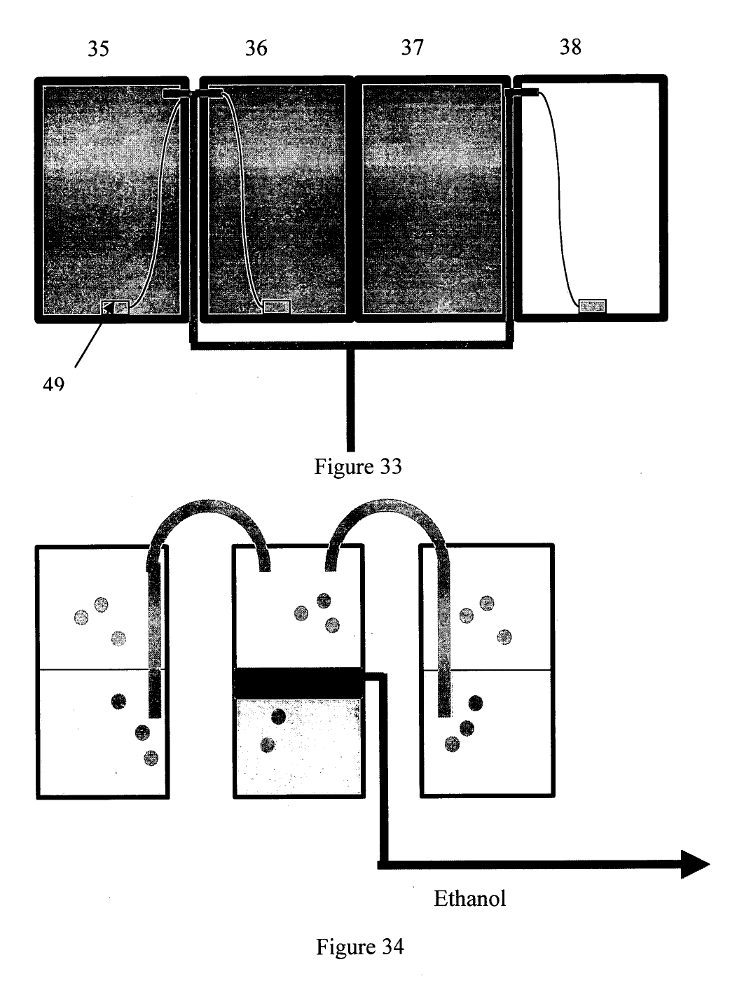

FIG. 33 shows another detail of the design setup showing flue gas bubbled through panels 35, 36, and 38 via an aerator (49). The aerator was a 3 W aquarium pump that allowed sufficient airflow for more than the 4 panels with a plastic airline. On the end of the airline was standard aquarium air bubbling stone. This released small bubbles. On the surface of the panel the bubbles had a scrubbing action which kept the surface clear of algal build-up. This is preferred in that any build-up can decrease the transmission of light and reduce the density of the algal mass on the bottom of the tank as disclosed below.

The algae that was fed this effluent flourished at a higher rate than the other samples fed distilled nutrient broths. After lipid extraction, the remaining algal mass is a useful animal feedstock. Copper is good for beasts and the algal slurry is protein-rich.

The algae in the tank that was exposed to the effluent flourished at a higher rate than the other panels which were not given effluent. Several visual observations indicated that the nitrates and phosphates were utilized by the algae for growth. Further, the reservoir used to recycle the water to the panel had a thick film of what looked like lipid rich material (probably the remaining material from the effluent).

The aerator (FIG. 7) had the tendency to move back and forth across the tank. This created a larger area being scrubbed. The tube was covered in a material made from the fuzzy side of VELCRO. This further allowed for automated scrubbing of the tank between cleaning. As shown in FIG. 7, an improved system that resulted in unexpected increased productivity was to increase the travel area of the bubbles. This has two advantages. The first is that the distance in which the CO₂ bubbles travel is greatly increased and the second is that the area along the pathways is clear of algal build-up.

One other embodiment that greatly increases the travel distance of the air hose is cycling the airflow to the algae. If the air (or flue gas) is cycled, water travels up the air hose making it heavy. This causes it to drop to the bottom of the tank, when air is sent through the air hose it again rises. We have found that shutting off the air, even once or twice an hour, keeps most of the tank clean.

Maximum travel distances on the airline hose are achieved by very small diameter tubing. In that the actual volume of CO₂ passing through the reactor is relatively small, this is not an impediment.

One other observation is that the algae only collects on the surface of the panel exposed to light. Thus, if the panel is at even a slight angle to the sunlight, it will assist in the scrubbing process as the CO₂ will travel on the sun exposed side and keep the surface clean.

### Density at Bottom of Tank

One of the problems associated with algaculture is that the ratio of algae to water is so low that a lot of energy has to be used to get sufficient algae to produce biofuels. Further, existing tubular bioreactors are not designed for the efficient harvesting of algae clumps. As a result, these tubular designs are not suited to certain species of algae that have the tendency to clump. One species in particular, Scenedesmus dimorphus has a very high lipid content but its use is usually avoided due to its clumping qualities.

The panels and the systems disclosed herein lends themselves to this species. During the night, the bubbler and pumps may be stopped. This can reduce the amount of agitation in the non-productive dark hours, allow the heavier clumps to drop to the bottom and reduce the energy requirements of the system. In the morning the clumps can be collected by the recirculating pump resulting in a higher ratio of lipid containing algae to water and consequently reduce the amount of energy required to obtain the oil.

### Density at Top of Tank

In the distillery, the water re-circulator filled the tank and the overflow drained into the reservoir. After the tests were concluded, the algae density in the reservoir was very high. This is because the CO₂ bubbles attached to some of the clumps and caused them to rise to the top of the tank. In the daytime, this may be the preferred method of harvesting, while at night we suck from the bottom.

### Panel Size

It was discovered that size is important to the lifespan of the panel. This was unexpected. A one square meter panel that had an airgap of 22 mm was tested. Upon filling the panel with water, the weight of the water caused the panel to distort and twist. The panel ended up bowing in the middle to over 50 mm. There was also considerable distortion of the panel. Although it held together, it would not be viable in a commercial environment. The stress would make the panel susceptible to breakage in the event of even a small impact. Further, the seals would eventually fail. When smaller panels (500 mm×1000 mm) were used, they didn't distort or bow significantly, in particular, when laid on their side.

### Example II

### Laboratory Testing of Algae Grown with Effluent

In these experiment, two batches of effluent were tested for effects upon algae growth in Test panels. The results are shown in Table 2.

TABLE 2

| | Batch 1 | Batch 1 | | Post | | | Food | Net | |
|---|---|---|---|---|---|---|---|---|---|
| Element | Sample 1 | Sample 2 | Average | Dilution | Batch 2 | | added | Change | Notes |
| Na ppm | S | S | S | | Na ppm | 44.66963 | 400 | Unknown | |
| Mg ppm | 34.42 | 66.84 | 50.63 | 5.06 | Mg ppm | 9.23956 | 300 | −304.18 | |
| P ppm | 186.49 | 322.81 | 254.65 | 25.4 | P ppm | 9.926043 | 160 | −144.53 | |
| S ppm | 25.31 | 55.43 | 40.37 | 4.03 | S ppm | 6.210393 | | 2.18 | (from flue gas) |
| K ppm | 202.05 | 354.96 | 278.5 | 27.8 | K ppm | 21.684 | 400 | −393.88 | |
| Zn ppb | 162.01 | 286.39 | 224.2 | 22.4 | Zn ppb | 31.92031 | | 9.52 | (from mineral water) |
| Ca ppm | 8.75 | 10.33 | 9.54 | 0.95 | Ca ppm | 34.77402 | | 33.82 | (from mineral water) |
| Mn ppm | 143.34 | 429.7 | 286.52 | 28.6 | Mn ppm | 61.04165 | 200 | −232.44 | |
| Fe ppb | 790.72 | 668.86 | 729.79 | 72.9 | Fe ppb | 51.32773 | 300 | −278.43 | |
| Cu ppm | 1.33 | 0.44 | 0.885 | 0.089 | Cu ppm | 0.01902 | | −0.07 | |
| Ni ppm | 0.006 | 0.008 | 0.007 | 0.00100 | Ni ppm | 0.011696 | | 0.01 | (from flue gas) |
| Cl ppm | Not Tested | N/A | N/A | | Cl ppm | 39.31347 | | Unknown | (from mineral water) |

1 litre of Batch 1 and 1 litre of Batch 2 of the effluent were mixed. Batch 1 is from the initial stages of the distillation process. All whiskies go through two stages of distillation. The first is referred to as the wash still, whilst the second distillation is commonly referred to as the spirit still. Over the course of a week the effluent tank will go through one wash still and one spirit still. It was therefore important to have samples from both batches, one from the wash still (Batch 1) and one from the spirit still (Batch 2) in order to ascertain how the algae would react in a production type environment.

The combined batches were then mixed with 18 litres of mineral water and added to the Test panel 2 resulting in a post dilution number in column five.

The sodium levels were great enough to be off the recordable level of the spectrometer, hence the “S” (saturated) and our inability to determine how much was consumed by the Test panel.

Plant food as listed in column eight was added.

The increase in sulphur levels are due to the flue gas. We suspect that we got some nickel from the flue gas as well, though we cannot confirm this. The calcium and zinc came from the mineral water. The copper levels dropped by quite a bit which implies that it is locked in the algae. We therefore intend to use the by-product after biofuel production as animal feed as copper is a common additive to animal feed.

No tests were conducted on control Panel 3 other than growth rates which are summarized below.

### Results

Algal Growth Rates
   1. Panel 1 received plant food and CO₂ from the flue gas.
   2. Panel 2 received plant food and CO₂ from the flue gas and 2 litres of effluent.
   3. Panel 3 had approx 2 litres which received only plant food
7 Day Growth Rates
   1. Panel 1—275 grams
   2. Panel 2—350 grams
   3. Panel 3—<10 grams
15 Day Growth Rates
   1. Panel 1—approx 300 grams—starting to crash due to lack of food
   2. Panel 2—1500 grams
   3. Panel 3—crashed, few viable cells

Algal growth for Panel 1 were above the norm compared to algae grown in a laboratory with ambient air pumped. Panel 2 which received the additional nutrients effluent had an average growth rate of 200 grams/m²/day (each panel was 0.5 m²). This is the highest figure attained to date and well above conventional photobioreactors.

In conclusion, the self-sustaining, self-powering systems disclosed herein provide inexpensive, simple and efficient systems for producing biodiesel in a carbon neutral manner, without consuming valuable and/or expensive resources. In addition, the system may be modified such that the product is ethanol or other alcohol fuel.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

### REFERENCES

Some relevant publications include the following (all of which are incorporated by reference in their entirely):
1. Mussgnug J H, Thomas-Hall S, Rupprecht J, Foo A, Klassen V, McDowall A, Schenk P M, Kruse O, Hankamer B.
Engineering photosynthetic light capture: impacts on improved solar energy to biomass conversion. Plant Biotechnol J. 2007 November; 5(6):802-14. Epub 2007 Aug. 31. PMID: 17764518
2. Chisti Y.
Biodiesel from microalgae. Biotechnol Adv. 2007 May-June; 25(3):294-306. Epub 2007 Feb. 13. Review. PMID: 17350212
3. Xu H, Miao X, Wu Q.
High quality biodiesel production from a microalga Chlorella protothecoides by heterotrophic growth in fermenters. J Biotechnol. 2006 Dec. 1; 126(4):499-507. PMID: 16772097
4. Miao X, Wu Q.
Biodiesel production from heterotrophic microalgal oil. Bioresour Technol. 2006 April; 97(6):841-6. Epub 2005 Jun. 4. PMID: 15936938
5. Lebeau T, Robert J M.
Diatom cultivation and biotechnologically relevant products. Part II: current and putative products. Appl Microbiol Biotechnol. 2003 February; 60(6):624-32. Epub 2002 Dec. 13. Review. PMID: 12664140
6. Batistella C B, Moraes E B, Maciel Filho R, Maciel M R.
Molecular distillation process for recovering biodiesel and carotenoids from palm oil. Appl Biochem Biotechnol. 2002 Spring; 98-100:1149-59. PMID: 12018237
7. Roessler P G, Bleibaum J L, Thompson G A, Ohlrogge J B.
Characteristics of the gene that encodes acetyl-CoA carboxylase in the diatom Cyclotella cryptica. Ann N Y Acad. Sci. 1994 May 2; 721:250-6. PMID: 7912057

1-36. (canceled)

37. A system for producing algal biomass, the system comprising (1) a first vessel containing a yeast and a hydrocarbon fuel source digestible by the yeast, wherein the yeast digests the fuel source to produce carbon dioxide; and (2) a second vessel comprising algae and water; whereby the first vessel is connected by a tube to the second vessel so as to allow passage of carbon dioxide from the first to the second chamber; wherein the carbon dioxide is used as a carbon source for the growing algae, and wherein the algal biomass is harvested from the second vessel.

38. The system of claim 37 wherein the hydrocarbon fuel source is selected from algal biomass, cellulose material, animal waste slurry, and an agricultural waste.

39. The system of claim 37 wherein the hydrocarbon fuel source comprises effluent from a malting and distilling process.

40. The system of claim 37 wherein the second vessel comprises at least two species of algae.

41. The system of claim 37 wherein the algae produce fatty acid-containing substances selected from triglycerides, phospholipids and fatty acid esters.

42. The system of claim 37 further comprising a biodiesel reaction chamber wherein the algal biomass is fed into the biodiesel reaction chamber to produce glycerol and biodiesel from a reaction between algal biomass and an alcohol in the presence of a base, and wherein the glycerol is used as a source of fuel to power various aspects of the system.

43. The system of claim 37, wherein said alcohol is ethanol, propanol, isopropanol, 1-butanol, 2-butanol or isobutanol.

44. The system of claim 37 wherein the energy output is at least 1.5 times the energy input.

45. The system of claim 37 wherein the algae comprise one or more of a blue-green alga, a cyanobacterium, a green alga, Chlorella, a green sulphur bacteria, a green non-sulphur bacteria, Euglena, a diatom, Cyclotella cryptica, and a micromonad.

46. The system of claim 37 wherein the system is carbon-negative.

47. A system for producing biodiesel, the system comprising (1) an anaerobic digestion chamber comprising a first micro-organism and at least one substrate, a fluid, and nutrients, (2) a bioreactor comprising a vessel containing algae, a fluid, and nutrients, wherein the anaerobic digestion chamber is in fluid communication with the bioreactor, wherein proliferation of the first organism generates a gas and an alcohol, wherein gas so generated is transferred to the bioreactor, whereby the algae in the bioreactor utilizes the gas to synthesize triglycerides and protein compounds, and further comprising (3) a biodiesel reaction chamber in which the triglycerides are converted into a biofuel by reaction with an alcohol in the presence of a base to produce methyl esters and glycerol, and wherein the glycerol is used as a source of fuel to power various aspects of the system.

48. The system of claim 37 wherein the system is a modular system comprising tile-shaped vessels shaped and adapted for exposing the algae to the sun, and wherein the vessels comprise two substantially flat sides and an airspace therebetween, and a cleaning device within the airspace, the vessel comprising at least one translucent side, an input aperture, an output aperture, and wherein the biomass substantially permeates the airspace, wherein the cleaning device impacts the travel time of carbon dioxide through the biomass, resulting in an improved growth rate of the biomass.

49. The system of claim 48 shaped and adapted for placement upon the surface of a building structure, a watercraft or an aircraft.

50. The system of claim 37 wherein the micro-organism has biochelatic properties.

51. The system of claim 37 wherein heavy metals are sequestered in the growing the algal biomass.

52. The system of claim 51 wherein the algae comprise cyanobacteria that bind copper using plastocyanin.
